(19) United States
(12) Patent Application Publication
(10) Pub. No.: US 2024/0109848 A1
(43) Pub. Date: Apr. 4, 2024

(54) PROCESS FOR THE PREPARATION OF IMIDAZOBENZODIAZEPINES

(71) Applicant: ODH IP Corp., NEW YORK, NY (US)

(72) Inventors: D. Tyler McQuade, Rockville, MD (US); Luke Rogers, Rockville, MD (US); Josef Maier, Rockville, MD (US); Julian Chesterman, Rockville, MD (US); Esther Chen, Rockville, MD (US); Saad Bux, Rockville, MD (US); Brenden Herrera, Rockville, MD (US); Loan Truong, Rockville, MD (US); Melanie Pachter, Rockville, MD (US)

(73) Assignee: ODH IP Corp., NEW YORK, NY (US)

(21) Appl. No.: 18/466,730

(22) Filed: Sep. 13, 2023

Related U.S. Application Data
(60) Provisional application No. 63/375,439, filed on Sep. 13, 2022.

(51) Int. Cl. C07D 243/28; C07D 243/30; C07D 487/04

(52) U.S. Cl. CPC C07D 243/28; C07D 243/30; C07D 487/04

(57) ABSTRACT

Described herein are methods for the preparation of imidazobenzodiazepines such as midazolam and its intermediates. The processes are capable of using continuous flow chemistry and systems.

### CROSS-REFERENCE TO RELATED APPLICATIONS

This application claims priority from Provisional Application No. 63/375,439, filed Sep. 13, 2022, the entire contents of which are hereby incorporated by reference.

### GOVERNMENTAL RIGHTS

This invention was made with government support under DARPA Cooperative Award no. HR-0011-16-2-0029 awarded by the Defense Advanced Research Projects Agency of the Department of Defense. The government has certain rights in the invention.

### FIELD OF THE INVENTION

The present disclosure provides improved processes for the preparation of imidazobenzodiazepines and their precursors, including midazolam and its precursors.

### BACKGROUND OF THE INVENTION

8-chloro-6-(2-fluorophenyl)-1-methyl-4H-imidazo[1,5-a][1,4]benzodiazopine

(midazolam), a pre-operative anesthetic, belongs to a class of imidazobenzodiazepine compounds which are useful as anticonvulsants, sedatives, and muscle relaxants.

The last step in the synthesis of midazolam is thermal decarboxylation of 8-chloro-6-(2-fluorophenyl)-1-methyl-4H-imidazo[1,5-a][1,4]benzodiazopine-3-carboxylic acid (tricyclic acid). Also produced in this step is 8-chloro-6-(2-fluorophenyl)-1-methyl-6H-imidazo[1,5-a][1,4]benzodiazopine (isomidazolam) and decomposition byproducts resulting from high temperature dehalogenation and dimerization of tricyclic acid. Removal of the decomposition byproducts and purification of midazolam and isomidazolam are accomplished by column chromatography (GB Patent 1,549,836 and U.S. Pat. Nos. 4,280,957, 4,440,685 and 4,377,523), a method which is impractical for large scale preparation of midazolam because of the costly chromatography equipment required.

Attempts at improving the overall yield of Midazolam have focused on isomerizing purified isomidazolam to midazolam by treatment of the former with potassium tert-butoxide in N,N-dimethylformamide (DMF) under kinetically controlled conditions (U.S. Pat. Nos. 4,377,523 and 4,440,685). This method is also impractical for large scale syntheses of Midazolam because of the amount of thermal energy required for removal of the DMF.

US2011/0275799A1 detailed an alternative process for the preparation of 4H-imidazo[1,5-a][1,4]benzodiazepines, through a new intermediate capable of at least partly overcoming the previously mentioned drawbacks with reference to the prior art. It was shown that performing the decarboxylation reaction on a derivative compound, 5-amino-methyl-1-phenyl-1 H-imidazole-4-carboxylic acid, prepared through acid hydrolysis of the imine, using an inorganic acid, gave a better yield of the product of formula (VI) thus avoiding the formation of isomers such as 6H-imidazole[l,5-a][1,4]benzodiazepines “isomidazolam” of formula (VIII):

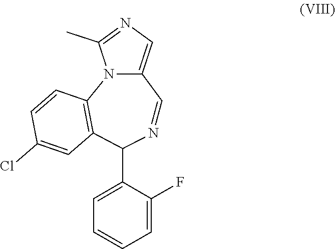 [SMILES: Cc1ncc2n1-c1ccc(Cl)cc1C(c1ccccc1F)N=C2]

However, even this method only produces ratios of midazolam:isomidazolam of about 9:1 or 10:1 when using hydrochloric acid in the decarboxylation reaction.

Thus, there is a continuing need in the pharmaceutical manufacturing industry for a large scale conversion of tricyclic acid to midazolam which minimizes isomidazolam formation and provides for non-chromatographic removal of byproducts.

### SUMMARY OF THE INVENTION

This patent disclosure provides an efficient process approach for midazolam

production and minimum formation of isomidazolam. Midazolam and its synthetic intermediates can be readily prepared with the method disclosed herein. It was surprisingly found that by using a melting solid acid reagent in the decarboxylation step, it avoids using any solvents such as N-methyl-2-pyrrolidone (NMP) or other high boiling solvents that are difficult to remove during purification. The melting solid acid is easily washed away during liquid/liquid extraction.

Moreover, with this inventive process, isomidazolam production is greatly reduced, providing ratios of midazolam:isomidazolam of about 180:1 or greater.

In accordance with a first aspect, the present invention provides a process for making a compound of formula II comprising the use of a continuous flow process as depicted in FIG. 1.

In accordance with a second aspect, the present invention provides a process for making a compound of formula III (midazolam-AFP).

In accordance with a third aspect, the present invention provides a process for making the compound of formula IV.

In accordance with a fourth aspect, the present invention provides a process for making the compound of formula V.

In accordance with a fifth aspect, the present invention provides a process for making the compound of formula VI (midazolam).

### BRIEF DESCRIPTION OF THE FIGURES

The following drawings form part of the present specification and are included to

further demonstrate certain aspects of the present disclosure. Certain aspects can be better understood by reference to one or more of these drawings in combination with the detailed description of specific aspects presented herein.

FIG. 1 illustrates a process flow diagram for the continuous synthesis of the compound of formula II.

FIG. 2 depicts a scheme of an aspect of the inventive process for preparation of midazolam (formula VI).

FIG. 3 depicts a scheme for preparation of midazolam (formula VI) reported before the invention was made.

FIG. 4 depicts a scheme of an aspect of the inventive process for preparation of midazolam (formula VI).

FIG. 5 depicts a scheme showing a synthetic route to Gly-NCA and lactam (formula II).

FIG. 6 depicts a process flow diagram of the continuous flow synthesis of lactam (formula II) using the scheme depicted in FIG. 5.

FIG. 7 shows HPLC chromatograms of each sampling point in the process. The double GLY-NCA addition impurity is denoted by the asterisk.

FIG. 8 depicts krapcho decarboxylation and imidazole cyclization for the gram scale process (top) and the manufacturing scale process (bottom).

FIG. 9 are overlayed extracted ion chromatograms of m/z 398, 400, and 402 for (top) the cascade process with NaBr, and (bottom) the two-step reaction with NaOH in ethanol.

FIG. 10 shows structures of midazolam carboxylates and their relative mother liquor concentrations at various pH values.

FIG. 11A shows 1H NMR spectra of midazolam carboxylic acid undergoing ring closure in DMSO-d6.

FIG. 11B shows a TGA plot of midazolam carboxylic acid isolated at pH 1 (green) and pH 5.5 (blue).

FIG. 12 shows crude LC chromatograms of decarboxylation reactions to midazolam. LCAP of midazolam and isomidazolam are displayed on the plots.

FIG. 13 shows a TGA plot comparing the decomposition rates of midazolam (green), PTSA (blue), and the midazolam:PTSA ion pair (light blue) at 200° C.

FIG. 14 depicts structures of midazolam carboxylate starting materials used. Panel A shows LC of starting materials. Panel B shows LC of reaction mixtures after heating with PTSA at 140° C. for 90 min. Panel C shows LC of reaction mixtures after heating with PTSA at 205° C. for 45 min. Panel D shows LC of reaction mixtures after heating with PTSA at 205° C. for 180 min.

FIG. 15 depicts a process flow diagram for kilogram scale manufacturing of midazolam.

### DETAILED DESCRIPTION

In the following sections, certain agents, methods, and processes of use to

prepare the disclosed compounds and compositions containing at least these compounds disclosed herein are described to detail certain aspects of the instant disclosure. It is understood that combinations, subsets, interactions, agents disclosed herein where specific reference of each individual and collective combinations and permutation of these compounds cannot be explicitly disclosed, each is contemplated. Additional aspects of the disclosure are described below.

It will be obvious to one skilled in the art that practicing the certain aspects does not require the employment of all or even some of the specific details outlined herein, but rather that concentrations, times, and other specific details can be modified through routine experimentation. In some cases, well known methods, or components have not been included in the description.

Aspects disclosed herein relate to novel chemical processes of use to synthesize imidazobenzodiazepines and their intermediates with a high level of purity. Importantly, the novel chemical processes disclosed herein can be used to synthesize 4H-imidazo[1,5-a][1,4]benzodiazepine compounds substantially free of their 6H-imidazo[1,5-a][1,4]benzodiazepine isomers. In some aspects, the processes of the instant disclosure can be used to synthesize midazolam substantially free of isomidazolam.

The term “4H-imidazo[I,5-a][1,4]benzodiazepines” or, more simply, “imidazobenzodiazepines”, are a class of benzodiazepines having the general formula (VII):

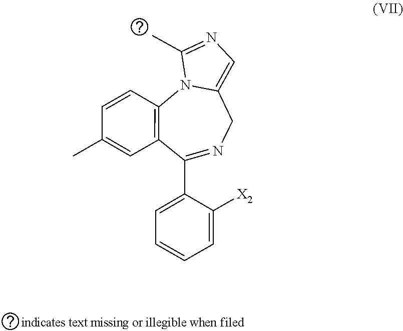

wherein the 1,4-diazepine ring is fused with a 1,3-imidazole ring. Midazolam is one of a number of several psychoactive isomers, which also include claimazolam, imidazenil, 1-hydroxy-midazolam, and desmethylmidazolam. It will be understood by those of skill in the art that the methods disclosed here can apply to the synthesis of any imidazobenzodiazepines in the class.

In some aspects, the present invention provides an on-demand, continuous method for the efficient synthesis of midazolam and its intermediates. As a result, the production is more efficient and less time-consuming than many conventional approaches. Meanwhile, in some aspects, the production can be accomplished in a portable and automated device, where different components are inter-connected and operated via a computerized system.

### I. Processes for Preparing Imidabenzodiazepines

One aspect of the instant disclosure encompasses a continuous flow process for synthesizing a compound of formula XVII,

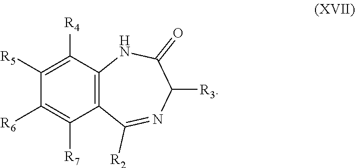

the process comprises reacting Glycine-N-Carboxyanhydride (Gly-NCA) with a phenone compound of formula XVIII in an acidic organic solution in a plug flow reactor;

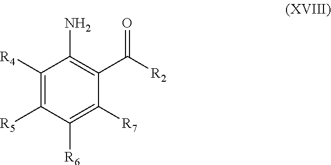

The process also comprises basifying the reaction product of the first step with a Lewis base in a stirred tank reactor to thereby result in lactam cyclization and formation of the compound of formula XVII.

In both compounds XVII and XVIII, R₂ and R₄-R₇ can each be independently selected from the group consisting of H, a halogen, an alkyl, an aryl, and a heteroaryl, wherein the aryl and the heteroaryl are each independently unsubstituted or are optionally independently substituted with one or more groups selected from the group consisting of alkyl, alkenyl, alkynyl, alkoxyl, alkylsulfo, alkylamino, halogen, thiol, hydroxyl, nitro, cyano, cycloalkyl, heterocyclic alkyl, aryl, heteroaryl, cycloalkoxyl, heterocyclic alkoxyl, cycloalkylthio, and heterocyclic alkylthio.

In some aspecxts, the acid organic solution comprises a non-polar solvent comprising an organofluorine acid. In some aspects, the acid organic solution comprises toluene comprising trifluoroacetic acid (TFA). In some aspects, the Lewis base is triethylamine (TEA).

The process can further comprise washing the reaction mixture with saturated aqueous alkali bicarbonate solution. In some aspects, the saturated aqueous alkali bicarbonate solution is saturated aqueous sodium bicarbonate.

In some aspects, the process further comprises separating organic and inorganic fractions of the reaction mixture. For instance, separating the organic and inorganic fractions of the reaction mixture can comprise passing the reaction mixture through a membrane separator. In some aspects, the membrane separator is a Zaiput membrane separator with an OB 900 filter.

The process can further comprise crystallizing the compound of formula XVII by pumping the organic fraction into a crystallizer comprising n-heptane (anti-solvent).

Another aspect of the instant disclosure encompasses a process for synthesizing a compound of formula XIX,

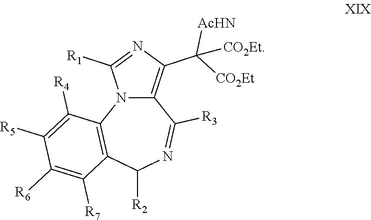

The process comprises mixing a compound of formula XVII

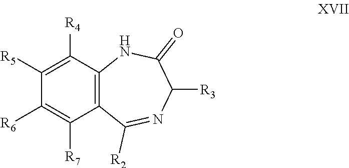

with a sufficient quantity of a polar, water miscible solvent; adding diethylacetamidomalonate, potassium tert-butoxide, and diphenylphosphorylchloride (DPPC) or dimorpholinophosphinyl chloride (DMPC); and allowing the reaction to proceed a sufficient period of time to produce the compound of formula XIX.

In both compounds XVII and XVIII, R₁-R₇ are each independently selected from the group consisting of H, a halogen, an alkyl, an aryl, and a heteroaryl, wherein the aryl and the heteroaryl are each independently unsubstituted or are optionally independently substituted with one or more groups selected from the group consisting of alkyl, alkenyl, alkynyl, alkoxyl, alkylsulfo, alkylamino, halogen, thiol, hydroxyl, nitro, cyano, cycloalkyl, heterocyclic alkyl, aryl, heteroaryl, cycloalkoxyl, heterocyclic alkoxyl, cycloalkylthio, and heterocyclic alkylthio.

The process also comprises allowing the reaction to proceed a sufficient period of time to produce the compound of formula XIX;

In some aspects, the compound of formula XVII is mixed with the polar, water miscible solvent in a reactor at a temperature of about 0° C., with stirring. In some aspects, about two moles of potassium tert-butoxide are added. In other aspects, about 0.7 mole of DPPC is added. In some aspects, the reaction is incubated at 0° C. for about 1 hour.

In some aspects, the process can further comprise crystallizing the compound of formula XIX by the steps of (a) inducing crystallization of the compound of formula XIX by the addition of an acid; (b) allowing the crystallized compound of formula XIX to precipitate; and (c) collecting the precipitate. The filtrate can be collected by filtration.

In some aspects, the process further comprises washing the filtered precipitate with a mixture of isopropanol and water (1:1); and drying the precipitate.

Yet another aspect of the instant disclosure encompasses a process for making the compound of formula XX;

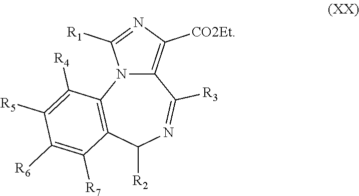

The process comprises mixing the compound of formula XIX with a sufficient quantity of the following: dimethyl sulfoxide (DMSO), an alcohol, and an alkali hydroxide and allowing the reaction to proceed for a period of time sufficient to synthesize the compound of formula XX; quenching the alkali hydroxide with a sufficient amount of acid; and heating the reaction mixture for a sufficient amount of time and a temperature to distill the alcohol off.

R1-R7 are each independently selected from the group consisting of H, a halogen, an alkyl, an aryl, and a heteroaryl, wherein the aryl and the heteroaryl are each independently unsubstituted or are optionally independently substituted with one or more groups selected from the group consisting of alkyl, alkenyl, alkynyl, alkoxyl, alkylsulfo, alkylamino, halogen, thiol, hydroxyl, nitro, cyano, cycloalkyl, heterocyclic alkyl, aryl, heteroaryl, cycloalkoxyl, heterocyclic alkoxyl, cycloalkylthio, and heterocyclic alkylthio.

In some aspects, the alkali hydroxide is sub-stoichiometric amounts of NaOH. In some aspects, the alcohol is ethanol. Distillation can be performed at 125° C. or more.

In some aspects, the process further comprises crystallizing the compound of formula XX by adding a sufficient quantity of water and cooling the reaction mixture with the addition of one or more seed crystals to create a crystallization slurry and collecting the crystallized compound of formula XX.

Another aspect of the instant disclosure encompasses a process for making the compound of formula XXI.

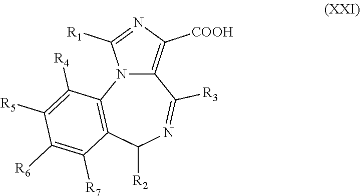

The process comprises mixing a compound of formula XX with an alcohol;

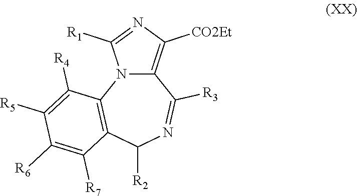

and adding a solution of aqueous alkali hydroxide.

The resultant solution is acidified with the addition of a sufficient quantity of inorganic acid with stirring to precipitate the compound of formula XXI resulting in a slurry; and collecting crystals formed by filtration and washing with a solution of about 1:1 acetone:water.

R1-R7 are each independently selected from the group consisting of H, a halogen, an alkyl, an aryl, and a heteroaryl, wherein the aryl and the heteroaryl are each independently unsubstituted or are optionally independently substituted with one or more groups selected from the group consisting of alkyl, alkenyl, alkynyl, alkoxyl, alkylsulfo, alkylamino, halogen, thiol, hydroxyl, nitro, cyano, cycloalkyl, heterocyclic alkyl, aryl, heteroaryl, cycloalkoxyl, heterocyclic alkoxyl, cycloalkylthio, and heterocyclic alkylthio.

One aspect of the instant disclosure encompasses a process for the preparation of 4H-imidazo[1,5-a][1,4]benzodiazepine compounds of formula IX or a salt thereof;

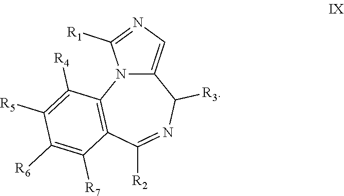

The process mixing a compound of formula X, a compound of formula XI, a compound of formula XII, or any combination thereof with about 3 to about 7 molar equivalents of a sulfonic acid to form a reaction mixture, wherein the compound of formula XI is a 6H-imidazo[1,5-a][1,4]benzodiazepine isomer of the compound of formula IX and the compounds of formula XII is a 6H-imidazo[1,5-a][1,4]benzodiazepine isomer of the compound of formula X:

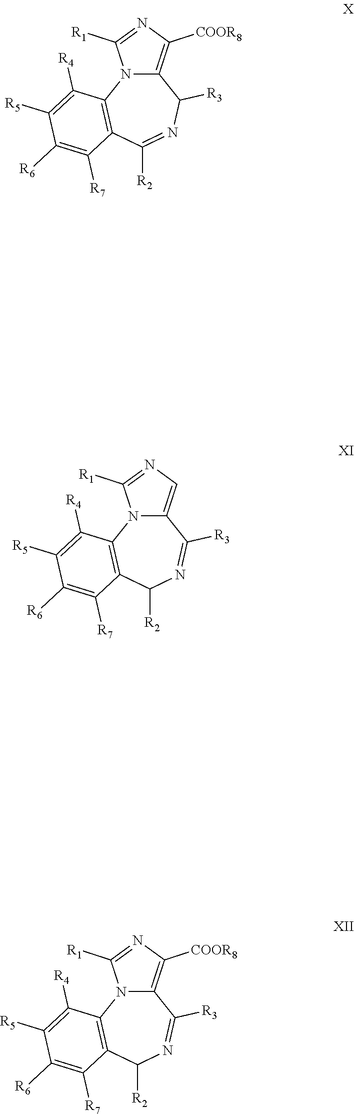

If the reaction mixture of comprises a compound of formula XI, a compound of formula XII, or any combination thereof, the process further comprises converting the 6H-imidazo[1,5-a][1,4]benzodiazepine isomer of formula XI to the 4H-imidazo[1,5-a][1,4]benzodiazepine isomer of formula IX to thereby synthesize the compound of formula IX and converting the 6H-imidazo[1,5-a][1,4]benzodiazepine isomer of formula XII to the 4H-imidazo[1,5-a][1,4]benzodiazepine isomer of formula X by heating the reaction mixture to a temperature ranging from about 130° C. to about 160° C. for a period of time ranging from about 60 minutes to about 120 minutes.

The process further comprises decarboxylating the 4H-imidazo[1,5-a][1,4]benzodiazepines of formula X and formula XII by heating any mixture from a) or b) comprising the 4H-imidazo[1,5-a][1,4]benzodiazepines of formula X and formula XII to a temperature ranging from about 170° C. to about 230° C. for a period of time ranging from about 30 minutes to about 240 minutes to thereby synthesize produce the 4H-imidazo[1,5-a][1,4]benzodiazepine of formula IX or a salt thereof.

R₁-R₈ are each independently selected from the group consisting of H, a halogen, an alkyl, an aryl, and a heteroaryl, wherein the aryl and the heteroaryl are each independently unsubstituted or are optionally independently substituted with one or more groups selected from the group consisting of alkyl, alkenyl, alkynyl, alkoxyl, alkylsulfo, alkylamino, halogen, thiol, hydroxyl, nitro, cyano, cycloalkyl, heterocyclic alkyl, aryl, heteroaryl, cycloalkoxyl, heterocyclic alkoxyl, cycloalkylthio, and heterocyclic alkylthio.

In some aspects, the 4H-imidazo[1,5-a][1,4]benzodiazepine compounds comprise compounds of formula XIII;

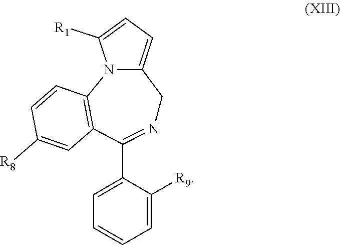

In such aspects, the process comprise: converting the 6H-imidazo[1,5-a][1,4]benzodiazepine isomer of formula XV to the 4H-imidazo[1,5-a][1,4]benzodiazepine isomer of formula XIII to thereby synthesize the compound of formula XIII and converting the 6H-imidazo[1,5-a][1,4]benzodiazepine isomer of formula XIV to the 4H-imidazo[1,5-a][1,4]benzodiazepine isomer of formula XVI; and decarboxylating the 4H-imidazo[1,5-a][1,4]benzodiazepines of formula XIV and formula XVI to thereby synthesize produce the 4H-imidazo[1,5-a][1,4]benzodiazepine of formula XIII or a salt thereof.

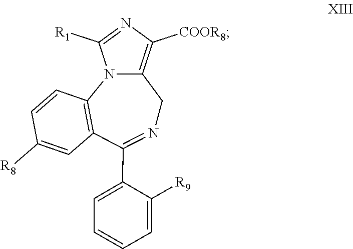

R₁ is selected from the group consisting of H, —CH₃, —CH₂OH, and —C₂H₅; and R₆ and R₉ are each independently selected from the group consisting of H and halogen; and R₈ is selected from —COOH and —CO2Et.

In some aspects, the 4H-imidazo[1,5-a][1,4]benzodiazepines comprise a compound of formula VI (midazolam);

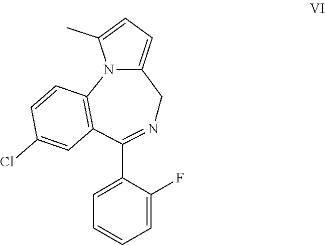 [SMILES: Cc1ccc2n1-c1ccc(Cl)cc1C(c1ccccc1F)=NC2]

wherein the 6H-imidazo[1,5-a][1,4]benzodiazepine isomer of midazolam (isomidazolam) is a compound of formula XXII

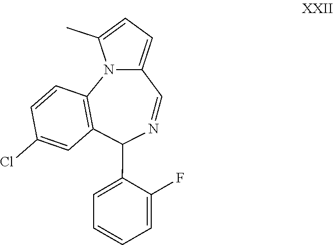 [SMILES: Cc1ccc2n1-c1ccc(Cl)cc1C(c1ccccc1F)N=C2]

and wherein the ratio of midazolam to isomidazolam is about 180:1 or greater.

In some aspects, the sulfonic acid is benzenesulfonic acid, para-toluenesulfonic acid (PTSA), methanesulfonic acid (MSA), or any combination thereof.

The process can further comprise purifying the 4H-imidazo[1,5-a][1,4]benzodiazepine compounds using the steps of: (a) adding a sufficient quantity of water to solubilize the 4H-imidazo[1,5-a][1,4]benzodiazepine of formula IX; (b) basifying the solution of a) with a sufficient quantity of concentrated aqueous alkali hydroxide solution; (c) extracting the solution of b) with a water-immiscible organic solvent at least two times, and collecting the organic layers; (d) treating the collected organic layers of c) with a sufficient quantity of activated charcoal for about 20 to 40 minutes; (e) filtering the charcoal treated organic solution through celite to remove the charcoal; and (f) concentrating the resultant solution of e) under vacuum to obtain the compound of formula IX as a solid.

In other aspects, the process further comprises purifying the 4H-imidazo[1,5-a][1,4]benzodiazepine compounds using the steps of: (a) adding a sufficient quantity of acetonitrile to solubilize the 4H-imidazo[1,5-a][1,4]benzodiazepine of formula IX; (b) cooling the solution of a) and seeding with crystals of the compound of formula IX; (c) collecting the resulting crystals by filtration; and (d) washing the crystals of c) in an isopropanol:n-heptane solution followed by n-heptane and drying the crystals.

Yet another aspect of the instant disclosure encompasses a process for the preparation of 4H-imidazo[1,5-a][1,4]benzodiazepine compounds of formula IX or a salt thereof;

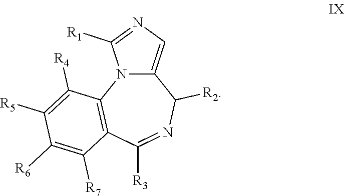

The process comprises combining one or more processes described herein above for synthesizing a compound of formula XVII, a compound of formula XIX, a compound of formula XX, compound of formula XXI or any combination of these processed with the process of synthesizing 4H-imidazo[1,5-a][1,4]benzodiazepine compounds of formula IX described herein above.

In some aspects, the process comprises combining one or more processes for synthesizing a compound of formula XVII, a compound of formula XIX, a compound of formula XX, a compound of formula XXI or any combination of these processes with the process of synthesizing 4H-imidazo[1,5-a][1,4]benzodiazepine compounds of formula IX in series. In other aspects, the process comprises combining one or more processes for synthesizing a compound of formula XIX, a compound of formula XX, a compound of formula XXI or any combination of these processes with the process of synthesizing 4H-imidazo[1,5-a][1,4]benzodiazepine compounds of formula IX in series. In other aspects, the process comprises combining one or more processes for synthesizing a compound of formula XX, a compound of formula XXI or any combination of these processes with the process of synthesizing 4H-imidazo[1,5-a][1,4]benzodiazepine compounds of formula IX in series. In other aspects, the process comprises combining one or more processes for synthesizing a compound of formula XXI with the process of synthesizing 4H-imidazo[1,5-a][1,4]benzodiazepine compounds of formula IX in series.

### II. Processes for Preparing Midazolam and Intermediates

One aspect of the instant disclosure encompasses a process for making a compound of formula II, an intermediate in the process for synthesizing midazolam:

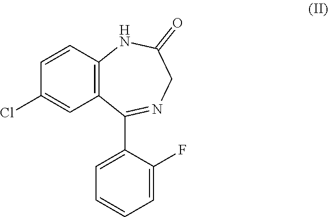 [SMILES: O=C1CN=C(c2ccccc2F)c2cc(Cl)ccc2N1]

The process comprises the steps of: (a) making a solution of toluene, trifluoracetic acid (TFA), and about 0.5 mole of 2-amino-5-chloro-2′-fluorobenzophenone; (b) stirring the mixture of a) and adding about 1.1 mole equivalents of glycine N-carboxyanhydride; (c) pumping the solution of b) into a plug flow reactor heated to about 50° C. with a flow rate of about 8 to 15 ml/min for about 10-20 minutes residence time; (d) converging the first outlet stream flow from c) with a second stream flow of triethylamine flowing at a rate of between about 0.7 to about 1.5 ml/min into a tank reactor heated to about 75° C. for about 90 minutes; (e) pumping the reaction solution of d) out of the reactor at a rate of about 10 to about 15 ml/min as a second outlet stream flow; (f) converging the second outlet stream flow of e) with a stream of saturated aqueous alkali bicarbonate solution at a flow rate of about 15 to about 25 ml/min; (g) separating the organic and inorganic fractions by combining the streams of f) into a membrane separator and collecting the organic layer created or in a gravity separator; (h) pumping the organic layer of g) into a crystallizer together with n-heptane (anti-solvent) at a flow rate of about 0.5 to 1.5 ml/min; and (i) collecting and drying the crystallized product.

Another aspect of the instant disclosure encompasses a process for making a compound of formula Ill (midazolam-AFP)

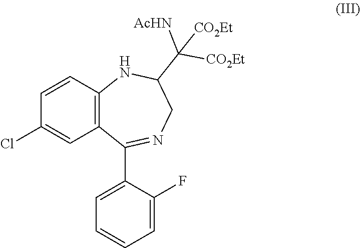 [SMILES: CCOC(=O)C(NC(C)=O)(C(=O)OCC)C1CN=C(c2ccccc2F)c2cc(Cl)ccc2N1]

The process comprises the steps of: (a) adding to a reactor at a temperature of about 0° C., a sufficient quantity of a polar, water miscible solvent with stirring; (b) adding to the solvent of a) about a mole of diethylacetamidomalonate, and about 0.6 mole of compound of formula II (midazolam-lactam or norflurazepam) to create a solution with stirring; (c) adding to the solution of b) about two moles of potassium tert-butoxide over a sufficient time to keep the temperature of the solution at about 0° C.; (d) adding to the solution of c) about 0.7 mole of diphenylphosphorylchloride (DPPC); and (e) allowing the reaction to proceed for about 1 hour. In some aspects, at step d), dimorpholinophosphinyl chloride (DMPC) is alternatively added.

In some aspects, the process can further comprise the steps of determining the completeness of the reaction of (f) by taking a sample of the solution of (e) and measuring the quantity of yield of the compound of formula III in the solution of (e).

In some aspects, the process can further comprise the steps of inducing crystallization of the compound of formula III by adding a sufficient quantity of about a 5% acetic acid solution in water to the solution of e) to induce crystallization of the compound of formula III; allowing the crystallized compound of formula III to precipitate or by adding a sufficient quantity of water to the solution of e); and collecting the precipitate by filtration.

In some aspects, the process optionally comprises the steps of: washing the filtered precipitate of i) with a mixture of isopropanol and water (1:1); and drying the precipitate.

Another aspect of the instant disclosure encompasses a process for making the compound of formula IV

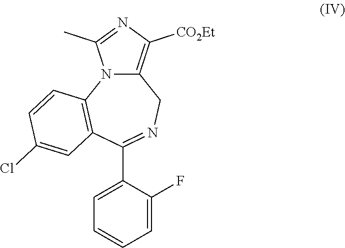 [SMILES: CCOC(=O)c1nc(C)n2c1CN=C(c1ccccc1F)c1cc(Cl)ccc1-2]

The process comprises the steps of: (a) adding about 2 moles of the compound of formula III into a reactor at a temp of about 25° C.; (b) adding to the reactor of a) a sufficient quantity of the following: dimethyl sulfoxide (DMSO), ethanol, and about 1.5 mol of alkali hydroxide, and stirring the resultant solution for about 30 to about 60 minutes; (c) optionally, determining the completeness of the reaction of b) by taking a sample of the solution of b) and measuring the quantity of yield of the intermediate formed midazolam-olefin intermediate (ethyl-2-acetamido-2-(7-chloro-5-(2-fluorophenyl)-1,3-dihydro-2H-benzo[e][1,4]diazepin-2-ylidene)acetate in the solution of b); (d) adding to the intermediate containing solution of b) a sufficient quantity, about 6 to 7 moles, of acetic acid and heating to 125° C.; (e) sparging the reactor with N₂ once the reactor reaches a temperature of at least about 70° C.; (f) stopping the N₂ sparging once the reactor reaches 125° C. and then stirring the reactor for between 30 to 60 minutes; (g) cooling the reactor to about 70° C.; (h) adding a sufficient quantity of deionized water and cooling the reactor to about 35° C. with the addition of one or more seed crystals to create a crystallization slurry; (i) cooling the slurry of h) to about −10° C. and stirring for at least about 30 min; (j) collecting the crystals of the compound of formula IV formed in step i) by filtration and washing with a sufficient quantity of deionized water; and (k) drying the crystals formed in j). At step b) an alkali ethoxide can be substituted for an alkali hydroxide.

Another aspect of the instant disclosure encompasses a process for making the compound of formula V

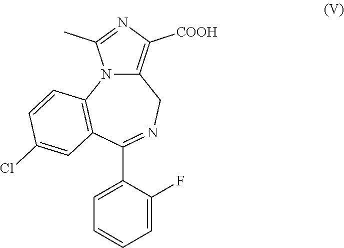 [SMILES: Cc1nc(C(=O)O)c2n1-c1ccc(Cl)cc1C(c1ccccc1F)=NC2]

The process comprises the steps of (a) adding to a reactor heated to about 50° C., a sufficient quantity of methanol with stirring; (b) adding to the methanol solution of a) about 1 to about 2 moles of the compound of formula IV and stirring the solution until the compound is dissolved; (c) adding to the solution of b) about 1 to 2 L of a about 2 to 3 molar aqueous alkali hydroxide solution and heating the reactor to about 70° C. for about 45 to 75 minutes; (d) optionally, determining the completeness of the reaction of c) by taking a sample of the solution of c) and measuring the quantity of yield of the compound of formula V formed in the solution of c); (e) acidifying the solution of c) with the addition of a sufficient quantity of 1M inorganic acid with stirring to precipitate the compound of formula V; (f) cooling the resulting slurry of e) to about room temp for about an hour and adjusting the pH of the slurry to about 5.2-5.7; (g) collecting the crystals formed in f) by filtration and washing with a solution of about 1:1 acetone:water; and (h) drying the crystals of the compound of formula V.

Yet another aspect of the instant disclosure encompasses a process for making the compound of formula VI

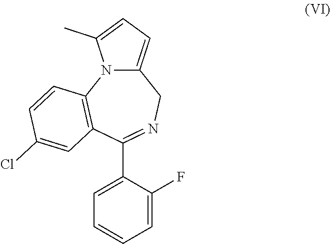 [SMILES: Cc1ccc2n1-c1ccc(Cl)cc1C(c1ccccc1F)=NC2]

The process comprises the steps of: (a) adding about 0.1 to 0.3 moles of the compound of formula V

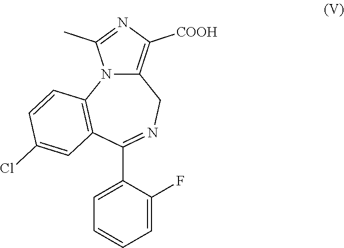 [SMILES: Cc1nc(C(=O)O)c2n1-c1ccc(Cl)cc1C(c1ccccc1F)=NC2]

to a container and adding about 5 to 6 mole equivalents of para-toluene sulfonic acid monohydrate and heating the container to about 150° C. for about 20 to 40 minutes to form a melted mixture; (b) heating the mixture of a) to 220° C. for about 2 to 3 hours to form a product; comprising the compound of formula VI; (c) cooling the reacted product of b) to about 100° C.; (d) adding to the mixture of c) a sufficient quantity of water to solubilize the product of c); (e) basifying the solution of d) with a sufficient quantity of concentrated aqueous alkali hydroxide solution; (f) extracting the solution of e) with a water-immiscible organic solvent at least two times, and collecting the organic layers; (g) treating the collected organic layers of f) with a sufficient quantity of activated charcoal for about 20 to 40 minutes; (h) filtering the charcoal treated organic solution through celite to remove the charcoal; and (i) concentrating the resultant solution under vacuum to obtain the compound of formula VI as a solid. At step a) the para-toluene sulfonic acid monohydrate can be substituted by another sulfonic acid such as benzene sulfonic acid monohydrate, para-toluene sulfonic acid (anhydrous), benzene sulfonic acid (anhydrous) or methane sulfonic acid.

In some aspects, the process can further comprise (j) dissolving the solids of step i) in a sufficient quantity of acetonitrile and heating the resulting solution to about 70° C. to produce an optically clear solution; (k) cooling the solution of j) to about 60° C. and seeding with crystals of the compound of formula VI; (l) cooling the solution of k) to about -2° C. and held at this temperature for about 30 mins; (m) collecting the resulting crystals by filtration; and (n) washing the crystals of m) in about a 1:4 isopropanol:n-heptane solution followed by n-heptane and drying under vacuum.

In some aspects, the process comprises: (j) at step d) diluting the cooled mixture of c) with water to a concentration of below about 40 mg of compound VI/ml; (k) filtering the solution of d) through a celite filter; (l) adding to the solution of e) about 6 mass equivalents (to midazolam) of acidic, neutral or basic alumina to create a slurry; (m) stirring the slurry of f) for about 1 to 2 hours and filtering out the alumina; (n) adding about 1/20 to ⅕ volumes of isopropanol and about 5 M aqueous alkali hydroxide is added to adjust the pH to greater than 10; (o) stirring the solution of h) for about 2 hours to allow the compound of formula VI to precipitate from the solution; (p) the resulting precipitate is redissolved in a sufficient quantity of 20% water in methanol and heated to about 40° C.; (q) the solution of j) is filtered to remove insoluble impurities and cooled to about 0° C. to allow crystallization; (r) collecting the resultant crystals from j) by filtration; (s)optionally washing the crystals with cold 20% water in methanol solution; and (t) drying the crystals of formula VI.

In some aspects, the solvent at step f) is selected from mixtures of water/methanol, water/isopropanol, ethyl acetate/water, acetonitrile/water, or ethyl acetate/n-heptane.

One aspect of the instant disclosure encompasses a process for the preparation of midazolam substantially free of isomidazolam. The process comprising combining one or more processes described herein above for preparing compounds of formulas III, IV, V, or any combination thereof, with the process of preparing the compound of formula VI. In some aspects, the process comprises combining the process of preparing compounds of formulas II, III, IV, V, and VI in series. In some aspects, the process comprises combining the process of preparing compounds of formulas III, IV, V, and VI in series. In some aspects, the process comprises combining the process of preparing compounds of formulas IV, V, and VI in series. In some aspects, the process comprises combining the process of preparing compounds of formulas V and VI in series.

### III. Aspects of Processes

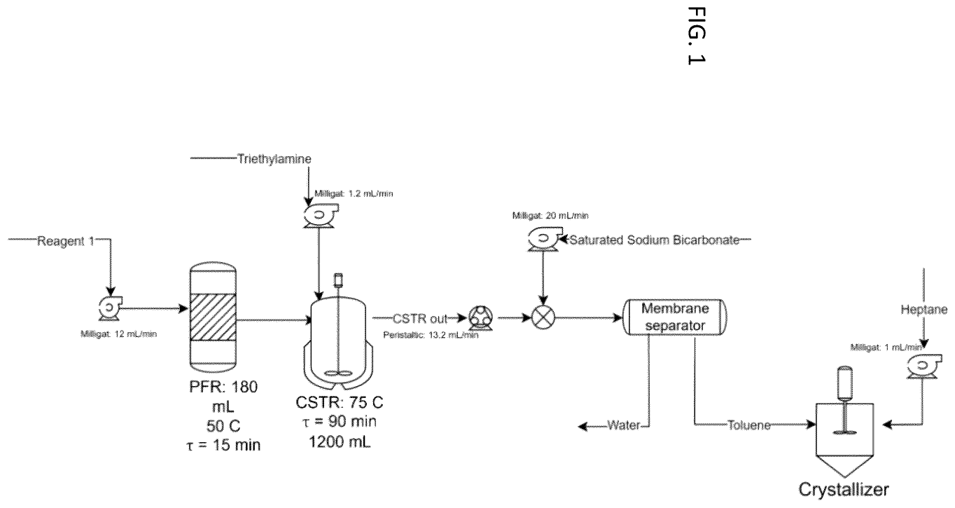

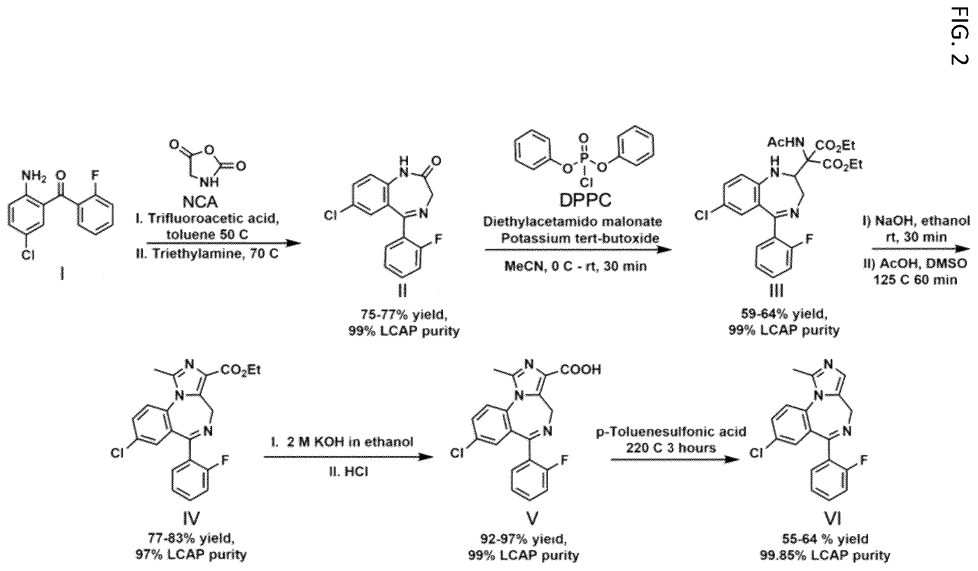

One aspect of the instant disclosure encompasses a process for making a compound of formula II comprising the use of a continuous flow process as depicted in FIG. 1. In an aspect the process comprises the steps of: a) making a solution of toluene, trifluoracetic acid, and about 0.5 mole of 2-amino-5-chloro-2′-fluorobenzophenone in a reagent container; b) stirring the mixture of a) and adding about 1.1 mole equivalents of glycine N-carboxyanhydride; c) pumping the solution of b) into a plug flow reactor heated to 50° C. with a flow rate of about 8 to 15 ml/min for about 10-20 minutes residence time; d) converging the first outlet stream flow from c) with a second stream flow of triethylamine flowing at a rate of between about 0.7 to about 1.5 ml/min into a tank reactor heated to about 75° C. for about 90 minutes; e) pumping the reaction solution of d) out of the reactor at a rate of about 10 to about 15 ml/min as a second outlet stream flow; converging the second outlet stream flow of e) with a stream of saturated aqueous alkali bicarbonate solution at a flow rate of about 15 to about 25 ml/min; g) combining the streams of f) into a membrane separator and collecting the organic layer created ; h) pumping the organic layer of g) into a crystallizer together with n-heptane (anti-solvent) at a flow rate of about 0.5 to 1.5 ml/min; and i) collecting and drying the crystallized product.

In accordance with a second aspect, the present invention provides a process for making a compound of formula III (midazolam-AFP) comprising the steps of: a) adding to a reactor at a temperature of about 0° C., a sufficient quantity of a polar, water miscible solvent with stirring; b) adding to the solvent of a) about a mole of diethylacetamidomalonate, and about 0.6 mole of compound of formula II (midazolam-lactam or norflurazepam) to create a solution with stirring; c) adding to the solution of b) about two moles of potassium tert-butoxide over a sufficient time to keep the temperature of the solution at about 0° C.; d) adding to the solution of c) about 0.7 mole of diphenylphosphorylchloride (DPPC) and e) allowing the reaction to proceed for about 1 hour.

In an alternative aspect, dimorpholinophosphinyl chloride (DMPC) can be used as an alternative DPPC.

In some aspects, the process further comprises the steps of: f) determining the completeness of the reaction of e) by taking a sample of the solution of e) and measuring the quantity of yield of the compound of formula III in the solution of e).

In another aspect, the process further comprises the steps of: g) adding a sufficient quantity of a 5% acetic acid solution in water to the solution of e) to induce crystallization of the compound of formula III or adding a sufficient quantity of water to the solution of e) to induce crystallization of the compound of formula III; h) allowing the crystallized compound of formula III to precipitate; and i) collecting the precipitate by filtration.

In a further aspect, the process optionally comprises the steps of j) washing the filtered precipitate of i) with a mixture of isopropanol and water (1:1) and k) drying the precipitate.

In accordance with a third aspect, the present invention provides a process for making the compound of formula IV comprising the steps of: a) adding about 2 moles of the compound of formula III into a reactor at a temp of about 25° C.; b) adding to the reactor of a) a sufficient quantity of the following: dimethyl sulfoxide (DMSO), ethanol, and about 1.5 mol of alkali hydroxide, and stirring the resultant solution for about 30 to about 60 minutes; optionally, c) determining the completeness of the reaction of b) by taking a sample of the solution of b) and measuring the quantity of yield of the intermediate formed midazolam-olefin intermediate (ethyl-2-acetamido-2-(7-chloro-5-(2-fluorophenyl)-1,3-dihydro-2H-benzo[e][1,4]diazepin-2-ylidene)acetate in the solution of b).

In an alternative aspect, at step b) an alkali ethoxide can be substituted for an alkali hydroxide.

In a further aspect, the process further comprises the steps of: d) adding to the intermediate containing solution of b) a sufficient quantity, about 6 to 7 moles, of acetic acid and heating to about 125° C.; e) sparging the reactor with N₂ once the reactor reaches a temp of at least 70° C.; f) stopping the N₂ sparging when the reactor temperature is about 125° C. and then stirring the reactor for between 30 to 60 minutes; g) cooling the reactor to about 70° C.; h) adding a sufficient quantity of deionized water and cooling the reactor to about 35° C. with the addition of one or more seed crystals to create a crystallization slurry; i) cooling the slurry of h) to about −10° C. and stirring for at least 30 min; j) collecting the crystals of the compound of formula IV formed in step i) by filtration and washing with a sufficient quantity of deionized water; k) drying the crystals formed in j).

In accordance with a fourth aspect, the present invention provides a process for making the compound of formula V comprising the steps of: a) adding to a reactor heated to 50° C., a sufficient quantity of methanol with stirring; b) adding to the methanol solution of a) about 1 to about 2 moles of the compound of formula IV and stirring the solution until the compound is dissolved; c) adding to the solution of b) about 1 to 2 L of a 2 to 3 molar aqueous alkali hydroxide solution and heating the reactor to 70° C. for about 45 to 75 minutes; optionally, d) determining the completeness of the reaction of c) by taking a sample of the solution of c) and measuring the quantity of yield of the compound of formula V formed in the solution of c); e) acidifying the solution of c) with the addition of a sufficient quantity of 1M acid, such as an inorganic acid like hydrochloric acid (HCl) with stirring to precipitate the compound of formula V; f) cooling the resulting slurry of e) to room temp for about an hour and adjusting the pH of the slurry to about 5.2-5.7; g) collecting the crystals formed in f) by filtration and washing with a solution of 1:1 acetone:water; and h) drying the crystals of the compound of formula V.

In accordance with a fifth aspect, the present invention provides a process for making the compound of formula VI comprising the steps of: a) adding about 0.1 to 0.3 moles of the compound of formula V to a container and adding 5 to 6 mole equivalents of para-toluene sulfonic acid monohydrate and heating the container to about 150° C. for about 20 to 40 minutes to form a melted mixture; b) heating the mixture of a) to about 220° C. for about 0.5 to 3 hours to form a product; comprising the compound of formula VI; c) cooling the reacted product of b) to about 100° C.; d) adding to the mixture of c) a sufficient quantity of water to solubilize the product of c); e) basifying the solution of d) with a sufficient quantity of concentrated aqueous alkali hydroxide solution; f) extracting the solution of e) with a water-immiscible organic solvent at least two times, and collecting the organic layers; g) treating the collected organic layers of f) with a sufficient quantity of activated charcoal for about 20 to 40 minutes; h) filtering the charcoal treated organic solution through celite to remove charcoal; and i) concentrating the resultant solution under vacuum to obtain the compound of formula VI as a solid.

In some aspects the para-toluene sulfonic acid monohydrate can be substituted by another sulfonic acid such as benzene sulfonic acid monohydrate, para-toluene sulfonic acid (anhydrous), or methane sulfonic acid.

In an optional aspect, the compound of formula VI of step i) can be further processed by the steps of: j) dissolving the solids of step i) in a sufficient quantity of about 4:1 methanol:water solution and heating the resulting solution to 50° C. to produce an optically clear solution; k) cooling the solution of j) to 40° C. and seeding with crystals of the compound of formula VI; l) cooling the solution of k) to 0° C. for about 2 hours; m) collecting the resulting crystals by filtration; and n) optionally washing the crystals of m) in a 6:4 methanol:water solution and drying under vacuum.

In an alternative fifth aspect, the process for making the compound of formula VI comprising at step d) diluting the cooled mixture of c) with water to a concentration of below about 40 mg of compound VI/ml; e) filtering the solution of d) through a celite filter; f) adding to the solution of e) about 6 mass equivalents (to midazolam) of acidic, neutral or basic alumina to create a slurry; g) stirring the slurry of f) for about 1 to 2 hours and filtering out the alumina; h) adding about 1/20 to ⅕ volumes of isopropanol and about 5M aqueous alkali hydroxide is added to adjust the pH to greater than 10; i) stirring the solution of h) for about 2 hours to allow the compound of formula VI to precipitate from the solution; j) the resulting precipitate is redissolved in a sufficient quantity of 20% water in methanol and heated to 40° C.; k) the solution of j) is filtered to remove insoluble impurities and cooled to 0° C. to allow crystallization; l) collecting the resultant crystals from j) by filtration; m) optionally washing the crystals with cold 20% water in methanol solution; and n) drying the crystals of formula VI.

In some aspects, the anti-solvent crystallization of the compound of formula VI (midazolam) can be performed with various mixtures of water/methanol, water/isopropanol, ethyl acetate/water, acetonitrile/water, or ethyl acetate/heptanes.

In alternative aspects, columns packed with activated carbon, activated charcoal, or activated alumina can be used instead of slurry mixtures of activated carbon, activated charcoal, or activated alumina during the purifications.

In some alternative aspects, for purification of the compound of formula VI (midazolam), extractions can alternatively be performed continuously using a continuous stirred tank reactor (CSTR) or in line via a static mixer apparatus.

In some alternative aspects, for purification of the compound of formula VI (midazolam), the separation step can be performed continuously using a gravity separator.

The melting solid acid prevents isomerization of midazolam to isomidazolam during the reaction, producing a ratio of at least 180:1, and in some cases up to 360:1.

It will be understood by those of ordinary skill in the art that the reagents and their quantities and the reaction times can vary within certain acceptable ranges

### Definitions

Unless defined otherwise, all technical and scientific terms used herein

have the meaning commonly understood by a person skilled in the art to which this invention belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them unless specified otherwise.

When introducing elements of the present disclosure or the preferred aspects(s) thereof, the articles “a”, “an”, “the” and “said” are intended to mean that there are one or more of the elements. The terms “comprising”, “including” and “having” are intended to be inclusive and mean that there may be additional elements other than the listed elements.

As used herein, the term “about,” can mean relative to the recited value, e.g., amount, dose, temperature, time, percentage, etc., ±10%, ±9%, ±8%, ±7%, ±6%, ±5%, ±4%, ±3%, ±2%, or ±1%.

As used herein, “analog,” refers to a chemical compound that is structurally similar to another compound (i.e., a so-called “reference” compound) but differs in composition, e.g., in the replacement of one atom by an atom of a different element, or in the presence of a particular functional group, or the replacement of one functional group by another functional group, or the absolute stereochemistry of one or more chiral centers of the reference compound. Accordingly, an analog is a compound that is similar or comparable in function and appearance but not in structure or origin to a reference compound.

As used herein, “isomers” refers to compounds having the same number and kind of atoms, and hence the same molecular weight, but differing in respect to the structural arrangement or configuration of the atoms.

As used herein, “alkyl” refers to a saturated aliphatic hydrocarbon group including C1-C20 straight chain and branched chain groups. Representative examples include, but are not limited to, methyl, ethyl, n-propyl, isopropyl, n-butyl, isobutyl, tert-butyl, sec-butyl, n-pentyl, 1,1-dimethyl propyl, 1,2-dimethyl propyl, 2,2-dimethyl propyl, l-ethyl propyl, 2-methylbutyl, 3-methylbutyl, n-hexyl, 1-ethyl-2-methylpropyl, 1,1,2-trimethylpropyl, 1,1-dimethylbutyl, 1,2-dimethylbutyl, 2,2-dimethylbutyl, 1,3-dimethylbutyl, 2-ethylbutyl, 2-methylpentyl, 3-methylpentyl, 4-methylpentyl, 2,3-dimethylbutyl, n-heptyl, 2-methylhexyl, 3-methylhexyl, 4-methylhexyl, 5-methylhexyl, 2,3-dimethylpentyl, 2,4-dimethylpentyl, 2,2-dimethylpentyl, 3,3-dimethylpentyl, 2-ethylpentyl, 3-ethylpentyl, n-octyl, 2,3-dimethylhexyl, 2,4-dimethylhexyl, 2,5-dimethylhexyl, 2,2-dimethylhexyl, 3,3-dimethylhexyl, 4,4-dimethylhexyl, 2-ethylhexyl, 3-ethylhexyl, 4-ethylhexyl, 2-methyl-2-ethylpentyl, 2-methyl-3-ethylpentyl, n-nonyl, 2-methyl-2-ethylhexyl, 2-methyl-3-ethylhexyl, 2,2-diethylpentyl, n-decyl, 3,3-diethylhexyl, 2,2-diethylhexyl, and the isomers of branched chain thereof. An alkyl group can be a lower alkyl having 1 to 6 carbon atoms. Representative examples include, but are not limited to methyl, ethyl, n-propyl, isopropyl, n-butyl, isobutyl, tert-butyl, sec-butyl, n-pentyl, 1,1-dimethylpropyl, 1,2-dimethylpropyl, 2,2-dimethylpropyl, 1-ethylpropyl, 2-methylbutyl, 3-methylbutyl, n-hexyl, 1-ethyl-2-methylpropyl, 1,1,2-trimethylpropyl, 1,1-dimethylbutyl, 1,2-dimethylbutyl, 2,2-dimethylbutyl, 1,3-dimethylbutyl, 2-ethylbutyl, 2-methylpentyl, 3-methylpentyl, 4-methylpentyl, 2,3-dimethylbutyl and etc. The alkyl group can be substituted or unsubstituted. When substituted, the substituent group(s) can have one or more groups independently selected from alkyl, alkenyl, alkynyl, alkoxyl, alkylsulfo, alkylamino, halogen, thiol, hydroxyl, nitro, cyano, cycloalkyl, heterocyclic alkyl, aryl, heteroaryl, cycloalkoxyl, heterocyclic alkoxyl, cycloalkylthio, heterocyclic alkylthio, carbonyl, carboxy or carboxylic ester.

As used herein, “cycloalkyl” refers to saturated and/or partially unsaturated monocyclic or polycyclic hydrocarbon group and have 3 to 20 carbon atoms. Representative examples of monocyclic cycloalkyl include, but are not limited to cyclopropyl, cyclobutyl, cyclopentyl, cyclopentenyl, cyclohexyl, cyclohexenyl, cyclohexadienyl, cycloheptyl, cycloheptatrienyl, cyclooctyl etc. A polycyclic cycloalkyl can include the cycloalkyl having Spiro ring, fused ring, and bridged ring.

As used herein, “heteroaryl” refers to an 5-14 membered aryl having 1 to 4 heteroatoms selected from O, S, and N as ring atoms, the remaining ring atoms being C. Examples of heteroaryl groups are furan, thiophene, pyridine, pyrrole, N-alkyl pyrrole, pyrimidine, pyrazine, imidazole, tetrazolyl, and the like. Heteroaryl herein can be fused to aryl, heterocyclic alkyl or cycloalkyl, wherein the ring connected with parent structure is heteroaryl. Heteroaryls herein can be substituted or unsubstituted. When substituted, the substituent group(s) can be one or more groups independently selected from of alkyl, alkenyl, alkynyl, alkoxyl, alkylsulfo, alkylamino, halogen, thiol, hydroxyl, nitro, cyano, cycloalkyl, heterocyclic alkyl, aryl, heteroaryl, cycloalkoxyl, heterocyclic alkoxyl, cycloalkylthio, heterocyclic alkylthio, carbonyl, carboxy or carboxylic ester.

As used herein, “hydroxyl” refers to an —OH group. As used herein, “hydroxyalkyl” refers to -alkyl-OH, wherein alkyl as defined above. As used herein, “halo” or “halogen” refers to fluoro, chloro, bromo, or iodo. As used herein, “thiol” refers to an organosulfur compound according to the form R—SH, where R represents an alkyl or other organic substituent. As used herein, “carbonyl” refers to —C(═O)—. As used herein, “nitro” refers to —NO2. As used herein, “cyano” refers to —CN. As used herein, “amino” refers to —NH2. As used herein, “carboxy” refers to —C(═O)OH. As used herein, “carboxylic ester” refers to —C(═O)O-alkyl.

As used herein, “optionally substituted” indicates that a group can be unsubstituted or can be substituted with one or more substituents as provided herein or known in the art. As used herein, “substituted” in reference to a group indicates that a hydrogen atom attached to a member atom within a group is replaced. It should be understood that the term “substituted” includes the provision that such substitution be in accordance with the permitted valence of the substituted atom and the substituent and that the substitution results in a stable compound (e.g. one that does not spontaneously undergo transformation such as by rearrangement, cyclization, or elimination). In certain aspects, a single atom can be substituted with more than one substituent as long as such substitution is in accordance with the permitted valence of the atom. Suitable substituents are defined herein for each substituted or optionally substituted group.

As used herein, “pharmaceutically acceptable” can refer to compounds, formulations, compositions, in any dosage form within the scope of sound medical judgment, suitable for use in contact with a human subject or tissues thereof and as appropriate, in animals without excessive toxicity, irritation, with reduced side effect or complication as a consumable or for administration thereof, commensurate with a reasonable benefit/risk ratio.

As used herein, “individual”, “subject”, “host”, and “patient” can be used interchangeably and refer to any subject or any mammalian regarding diagnosis, treatment, prophylaxis or therapy as desired; for example, humans (e.g., adults, adolescents, toddlers, senior adults, children, infants and a fetus), companion animals (e.g., pets, horses), livestock, or other animals.

As used herein, “treat,” “treating” or “treatment” can refer to administering an agent disclosed herein and reversing, ameliorating, or inhibiting onset or inhibiting progression or curing or placing into remission of a health condition or disease or a symptom of the health condition or disease (e.g., cancer).

As various changes could be made in the above-described processes without departing from the scope of the invention, it is intended that all matter contained in the above description and in the examples given below, shall be interpreted as illustrative and not in a limiting sense.

### EXAMPLES

All patents and publications mentioned in the specification are indicative of the levels of those skilled in the art to which the present disclosure pertains. All patents and publications are herein incorporated by reference to the same extent as if each individual publication was specifically and individually indicated to be incorporated by reference.

The publications discussed throughout are provided solely for their disclosure before the filing date of the present application. Nothing herein is to be construed as an admission that the invention is not entitled to antedate such disclosure by virtue of prior invention.

The following examples are included to demonstrate the disclosure. It should be appreciated by those of skill in the art that the techniques disclosed in the following examples represent techniques discovered by the inventors to function well in the practice of the disclosure. Those of skill in the art should, however, in light of the present disclosure, appreciate that many changes could be made in the disclosure and still obtain a like or similar result without departing from the spirit and scope of the disclosure, therefore all matter set forth is to be interpreted as illustrative and not in a limiting sense.

### Example 1. Preparation of the Compound of Formula II

To a 5 L bottle was added 3 L toluene, 160 mL trifluoroacetic acid, and 2-amino-5-chloro-2′-fluorobenzophenone (135.0 g, 0.541 mol). The mixture was stirred to a homogenous solution. Glycine N-carboxyanhydride (60.0 g, 0.595 mol, 1.1 equivalents) was added at once and the solution was pumped through a 180 mL plug flow reactor (PFR) heated at 50° C. with a flow rate of 12 mL/min for a 15 min residence time. The outlet stream converged with a stream of triethylamine flowing at 1.2 mL/min into a 2 L continuous stirred tank reactor (CSTR) heated at 75° C. After a 90 min residence time the reaction was pumped out of the CSTR at a rate of 13.2 mL/min where it met a stream of saturated aqueous sodium bicarbonate solution flowing at 20 mL/min. The combined stream was passed through a Zaiput SEP-200 membrane separator and the organic layer was collected in a crystallizer along with n-heptane anti-solvent flowing at 1 mL/min. The crystallized product was collected and dried yielding 106.0 g of midazolam lactam, a 75.6% yield based on 2.7 L of starting solution.

### Example 2. Preparation of the Compound of Formula III

To a 5-L reactor with a jacket temperature of 0° C. was added acetonitrile (1.6 L) with stirring. Next, diethylacetamidomalonate (262.9 g, 1.21 mol) and midazolam-lactam (norfluazepam) (175.0 g, 0.606 mol) were added to the reactor and stirred. Potassium tert-butoxide (218.4 g, 1.94 mol) was added to the reactor at 0° C. over 5 minutes. After the reactor had cooled back to 0° C., diphenylphosphoryl chloride (DPPC) (195.4 g, 0.727 mol) was added, and the reaction was stirred for 1 hour. Once the reaction was complete (determined by liquid chromatography mass spectrometry (LCMS)), 500 mL of a 5% acetic acid solution in water was added to the reactor to induce crystallization. After 45 min of stirring the precipitate was filtered and washed with 2 L of 1:1 isopropanol:water. The powder was dried in a vacuum oven to yield 188.8 g of an off-white powder, a 63.8% yield.

### Example 3. Preparation of the Compound of Formula IV

Compound III (0.995 kg, 1.957 mol) was added to a 5-L reactor equipped with stirring and sparging capabilities and an initial jacket temperature of 25° C. (room temperature). Dimethyl sulfoxide (DMSO) (1.194 L), ethanol (1.194 L), and dissolved sodium hydroxide (57.72 g, 1.47 mol) were then added into the reactor and stirred for 45 minutes. Once the midazolam-olefin intermediate (ethyl-2-acetamido-2-(7-chloro-5-(2-fluorophenyl)-1,3-dihydro-2H-benzo[e][1,4]diazepin-2-ylidene)acetate) was complete (determined by LCMS), acetic acid (0.381 L, 6.65 mol) and DMSO (0.591 L) were added to the reactor. The reaction was then heated to 125° C., with nitrogen sparging beginning once the reaction reached 70° C. Once at 125° C., the sparging was turned off and the reaction stirred for 45 minutes, at which point the reaction was complete (determined by LCMS). The reaction was cooled to 70° C. and deionized water (0.325 L) was added to the reactor to prepare the solution for crystallization. The reaction was then cooled to 35° C. and seed crystals (1.0 g) were added to begin crystallization. The crystallization slurry was then cooled to −10° C. After 30 minutes of stirring at −10° C., the crystals were filtered and washed with 60:40 DMSO: DI water (2.87 L) followed by a wash of DI water (1.91 L). The resulting solid was dried in a vacuum oven to obtain 624 g (80% yield) of an off-white/peach colored solid.

### Example 4. Preparation of the Compound of Formula V

To a 5-L reactor with a jacket temperature set to 50° C., was added methanol (2.8 L) with stirring. Next, ethyl-8-chloro-6-(2-fluorophenyl)-1-methyl-4H-benzo[f]imidazo[1,5-a][1,4]diazepine-3-carboxylate (TCE) (600.0 g, 1.508 mol) was added and the mixture was stirred until the solids were completely dissolved. An aqueous solution of 2.5 M potassium hydroxide (1.206 L, 3.015 mol) was added, and the reaction was heated to 70° C. and kept at that temperature for 1 hour. Once the reaction was complete (determined by LCMS), the reaction temperature was brought down to 50° C. before adding 1 M hydrochloric acid solution (2.57 L, 2.57 mol) slowly over a period of 10 minutes while stirring to start the precipitation. The slurry was set to cool down to room temperature and was stirred at room temperature for 1 hour. The pH of the slurry was adjusted by adding 20 mL portions of a 1 M hydrochloric acid solution until the final pH is ˜5.5. The slurry was filtered, and the solids were washed with 1.2 L of 1:1 acetone: water. The solids were dried in the oven at 105° C. until dry to obtain 535.5 g of an off-white powder with a yield of 96.0%.

### Example 5. Preparation of Midazolam (Compound VI)

Compound V (100.0 g, 0.270 mol) was added to a 1L round-bottom flask along with p-toluenesulfonic acid (p-TSA) monohydrate (300.0 g, 1.58 mol) (5.8 equivalents) and melted at 150° C. and held for 30 mins. The reaction was then heated to 220° C. After 2.5 hours reaction conversion was tested by LCMS and at this point the reaction was 96% converted to midazolam with minimal impurities. The reaction was then cooled to 100° C. before water addition. 200 mL water was carefully and slowly added to solubilize the melt and the reaction was then cooled to room temperature. Next, 300 mL of 10 M sodium hydroxide was added slowly to basify the reaction, at a rate that kept the temperature below 60° C. The aqueous solution was extracted twice with 700 mL ethyl acetate and the combined organic layers were pumped through a 30 g charcoal column at 15 mL/min (5 min residence time). The resulting decolorized solution was then concentrated under vacuum to yield 63 grams of crude midazolam powder. The powder was then dissolved in 250 mL of acetonitrile (250 mg/mL concentration) by heating to 70° C. The solution was slowly cooled to 60° C. and 500 mg of pure midazolam was added as a seed, and the solution was then cooled to −2° C. and held for 30 minutes. The resulting slurry was filtered, and the powder was washed with 20:80 (v/v) isopropanol/n-heptane (2×150 mL), followed by a (1×100 mL) displacement wash with n-heptane. The solids were then dried in a vacuum oven at 85° C. and -29 in Hg for 4 hours, then at 105° C. and 1 atm for 1.5 hours. Final dried weight was 48.5 g of white solids, a 55% yield.

### Example 6. Compound VI Synthesis With Alternative Crystallization (Methanol/Water Crystallization)

Compound V (50.0 g, 0.135 mol) was added to a 500 mL round-bottom flask along with p-toluenesulfonic acid monohydrate (150.0 g, 0.789 mol)(5.8 equivalents) and melted at 150° C. and held for 30 mins. The reaction was then heated to 220° C. After 2.5 hours reaction conversion was tested by LCMS and at this point the reaction was 86% converted to midazolam with minimal impurities. The reaction was then cooled to 100° C. before water addition. 100 mL water was added to solubilize the melt and the reaction was then cooled to room temperature. Next, 100 mL of 10 M sodium hydroxide (NaOH) was added slowly to basify the reaction. The aqueous solution was extracted twice with 400 mL ethyl acetate and the combined organic layers were treated with charcoal (33 g) for 30 mins at room temperature. The organic solution was filtered through celite to remove the charcoal and concentrated under vacuum to yield crude midazolam powder as a pale-yellow solid. The crude solid was dissolved in 140 mL methanol and 30 mL water and heated to 50° C. to produce a clear solution. The solution was cooled to 40 C and seeded with 2% of the expected yield of Midazolam and was then cooled to 0° C. over 2 hours and then held at this temperature for 1 hour. The resulting crystals were filtered and washed with 50 mL of cold 6:4 v:v methanol:water. The resulting white crystals were dried under vacuum to a dry weight of 23.5 g, a 53.4% yield.

### Example 7. Midazolam Alternative Purification

An alternative method dilutes the cooled melt reaction to a concentration of 40 mg/mL or lower. This solution is stirred for 5-30 minutes and then filtered through a Celite 545 plug to remove insoluble impurities. Activated alumina (acidic, neutral, or basic, 6 mass equiv. relative to midazolam) was added to the mother liquor and this slurry was stirred for 1.5 h at room temperature before removing the alumina via filtration. To the solution, isopropanol (IPA) was added (5-20% relative to the aqueous solution) and then 5 M NaOH was added to adjust the pH to over >10. This solution was then stirred at room temperature for 2 h to allow the product to precipitate from the solution. The collected powder was then redissolved in 20% water in methanol and heated to 40° C. This solution was filtered warm to remove insoluble impurities and then the solution was stirred in an ice bath to allow crystallization. The final solids were collected via filtration and displacement washed with cold water/methanol (20% water, 2 mL per g product).

### Example 8. Improved Process for Production of Midazolam

The Covid-19 pandemic of 2020 put a massive strain on the medical supply chain, causing drug shortages of critical care medications. Common ICU medicines such as sedatives, paralytics, and anesthetics were significantly affected due to their high demand for use on intubated Covid patients. For example, the drug product of active pharmaceutical ingredient (API) Midazolam, a benzodiazepine sedative on the World Health Organization's list of essential medicines, has been reported in shortage by the FDA since April of 2020, with a current estimated recovery in March 2024.

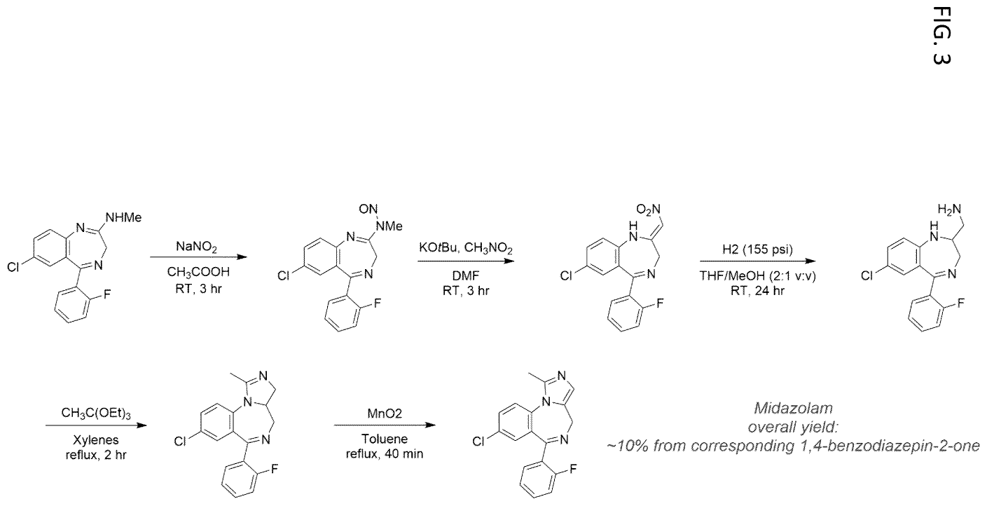

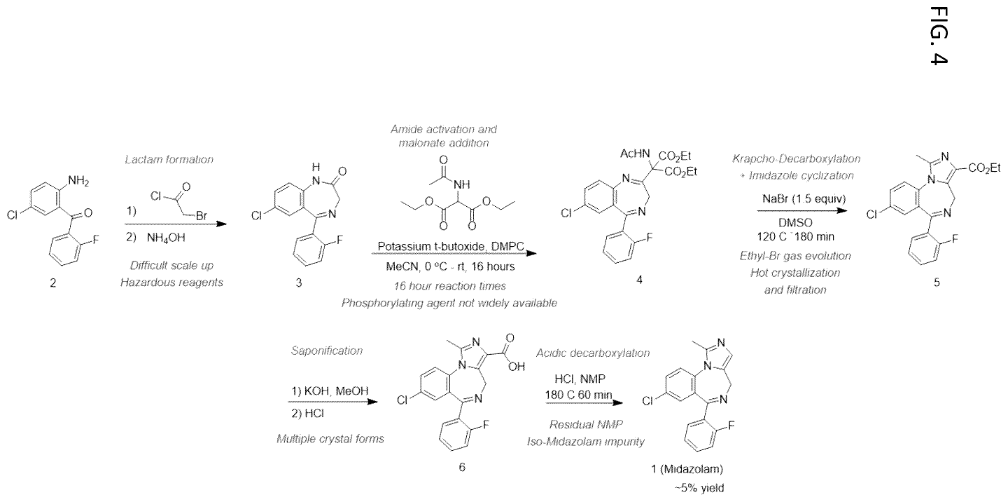

In this Example, the development of a manufacturing route to Midazolam is shown to relieve stress in the API supply chain, using continuous flow chemistry where applicable. The synthesis of Midazolam was first published by Hoffman-La-Roche in 1978 and is shown in the scheme of FIG. 3. In this synthesis, the fused imidazole ring is installed via carbon-carbon bond forming step between the activated diazepine starting material and nitromethane, followed by reduction, and acetylation of the primary amine. The cyclization reaction is performed by heating in polyphosphoric acid, followed by manganese oxide aromatization to the Imidazole to yield Midazolam with a 10.4% yield. Later work from Hoffman-La-Roche replaced N-nitrosomethylamino leaving group with an iminophosphate, which was shown to react with the anion of malonic-ester derivatives that led to carboxylic acid bearing imidazoben-zodiazapines (but did not use this method to synthesize midazolam) and did not require a final aromatization step. This strategy could create the carboxylic acid derivative of midazolam, with the final step being a decarboxylation. Abbot laboratories patented a one-step synthesis of Midazolam from this decarboxylation in 2003 which was carried out by heating to high temperatures in mineral oil. The reaction resulted in midazolam and isomidazolam at a 10:1 ratio (FIG. 4). Isomidazolam, a structural isomer of Midazolam, is a difficult to purge impurity that must be control below 0.1% in the drug substance per USP guidelines. Abbot laboratories reported that multiple crystallizations could re-move this impurity to give Midazolam product in greater than 99% purity. Therefore, our proposed route is a hybrid of the Hoffman-La Roche imidophosphate and Abbot laboratories' decarboxylation.

Development of this hybrid route was started on the gram scale using benzophenone (formula I) as a starting material. The amide bond formed between the aniline and acid chloride, followed by cyclization using ammonium hydroxide, resulted in lactam (formula II) in moderate yields. However, poor solubility of the amide intermediate caused thick precipitation, as well as safety concerns with the acid chloride reagent prevented easy scale up. The reaction of lactam (formula II) with di-morpholinophosphinyl chloride (DMPC) in the presence of base followed by the addition of diethylacetomidomalonate formed compound of formula III but in low yields and long reaction times. The morpholinophosphinyl chloride was initially used as a safer option to reported diethylphospinyl chloride, a nerve agent, however, this required the in-house synthesis of DMPC and showed much slower reaction times. The compound of formula 3 reacted with NaBr in hot DMSO in a cascade Krapcho/imidazole cyclization reaction. Despite full conversion, several biproducts were formed, including a cyclic urea that accounts for -10% of the mass balance, resulting in a difficult crystallization that was sensitive to oiling at temperatures lower than 50° C. and a final isolated yield of ˜40%. Final saponification followed by an acidic decarboxylation in NMP at high temperatures resulted in midazolam with moderate levels of iso-midazolam and required extensive purification and color removal. This initial route produced midazolam on the gram scale with an overall yield of ˜5%, a yield too low for the manufacturing route, so the overall yield needs to be significantly improved. Thus, the lowest yielding steps, the imidazole ring formation step, and the decarboxylation were targeted for improvement. Both steps were plagued with low purities, biproducts that were difficult to purge, and high-boiling water miscible solvents. Therefore, effort was focused on understanding and eliminating the numerous side-reactions and resulting biproducts to clean up the crude reaction mixtures and ease the purification load. Long reaction times and process safety of the first two steps were also addressed, which were solved by using continuous manufacturing to produce lactam (formula I). Additional details are provided in Examples 9-13 herein below.

### Example 9. Lactam Synthesis

One aim of the ultimate production of midazolam is the use of continuous manufacturing strategies where appropriate. In the case of lactam (formula II), this was demonstrated by using in series a plug flow reactor (PFR) and continuous stirred tank reactor (CSTR). This allows for greater reaction control, safety, and a straightforward scale-up. Researchers at Purdue University and Continuity Pharma recently demonstrated a novel 5-step continuous synthesis to a structurally similar benzodiazepine, loreazepam, on the gram scale. Like the discovery route depicted in FIG. 4, the reported synthesis utilized bromoacetyl chloride to create the amide, followed by cyclization to the lactam with ammonium hydroxide.

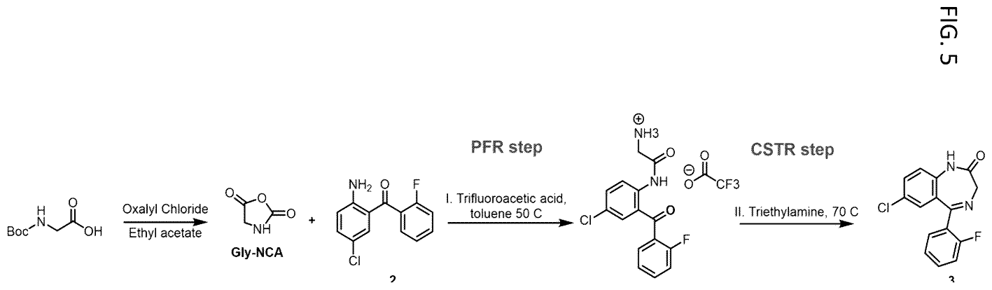

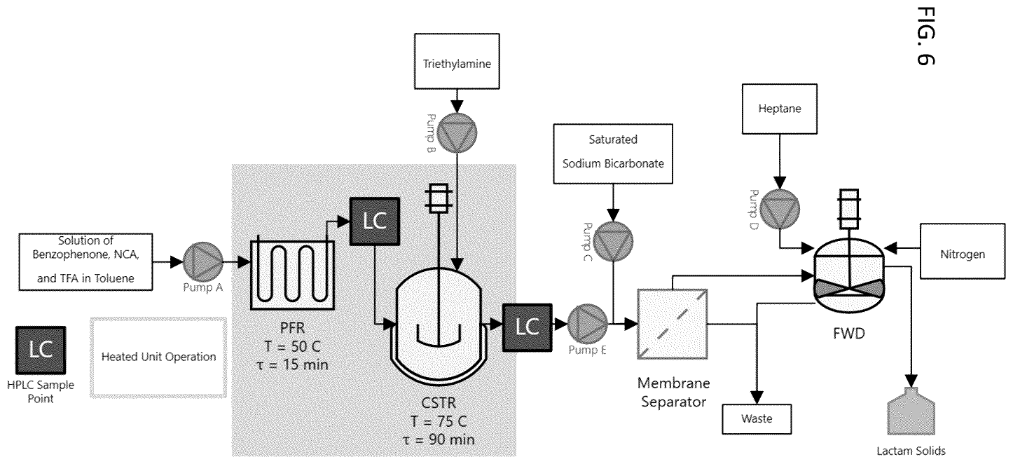

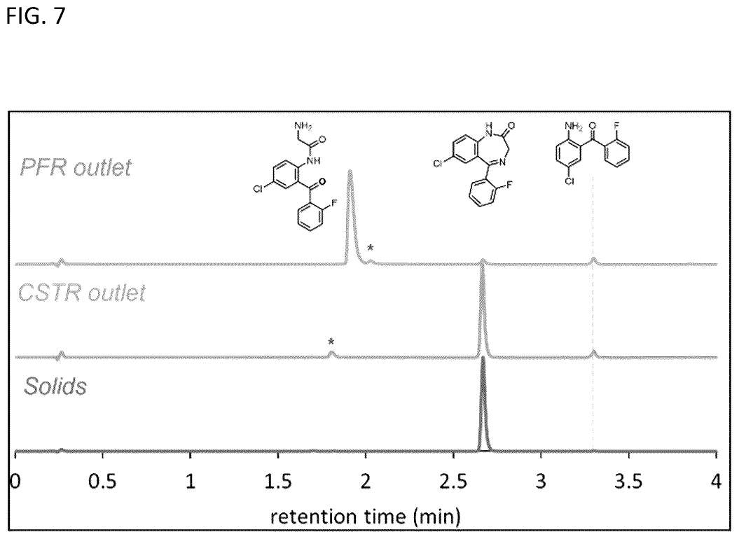

It was desirable to avoid using this sequence at scale due to solubility issues with the bromide intermediate. Therefore, synthesis of benzodiazepines from their corresponding aminobenzophenones using N-Carboxyanhydrides (NCA) as the electrophile was attempted. At room temperature, the NCAs can be prepared from N-Boc amino acids via PCI3 or Oxalyl Chloride. Therefore, the NCA was prepared from N-Boc glycine and oxalyl chloride in ethyl acetate on the 100 g scale and used this material as feedstock for a continuous flow reaction depicted in the scheme of FIGS. 5 and 6. A two-stage continuous process of amide-bond formation in acidic organic, followed by lactam ring formation in basic organic was envisioned. Solubility studies indicate toluene to be an ideal reaction solvent, and the lactam product showed lower solubility in toluene, which allows for crystallization with a low amount of anti-solvent from a toluene/heptane mixture (80:20 v:v)(see supporting information for solubility studies). Therefore, the reaction between Gly-NCA and benzophenone (formula I) occurred in toluene with trifluoroacetic acid (TFA). The feedstock was pumped through three 60 mL PFRs in series, at 50° C. and a 15 min residence time. Initial studies showed that TFA equivalence and temperature of the PFR were critical parameters for minimizing the formation of a second addition of NCA to the Amide intermediate. Basification with triethylamine (TEA) resulting in lactam cyclization was performed in a 1 L CSTR due to the precipitation of TEA salts. The suspension was then transferred via a peristaltic pump to a t-joint, where the reaction was washed with aqueous sodium bicarbonate to aid in salt removal, followed by passage through a Zaiput membrane separator with an OB 900 filter to separate the layers. The organic layer was then pumped into a crystallizer along with n-heptane, resulting in crystallization of the Lactam, 3. Samples were taken at each outlet and analyzed by LCMS to determine conversion and impurity profile (FIG. 7).

Near complete conversion to the amide was seen at the PFR outlet, with an LCAP purity of 90.5%, 3% conversion to the lactam, probably due to slight basification in the HPLC diluent, and 4% remaining starting material. The double GLY-NCA addition impurity, denoted by the asterisk, was held to 2.3% LCAP. Full ring closure to the Lactam was seen in the CSTR outlet, and the remaining impurity and starting material were purged in the crystallization, resulting in solids with >99% LCAP. Two demonstrations were performed with this set-up using 1 L and 2.5 L feedstocks, generating 40.3 and 106.0 g of product, respectively. Isolated yields of 76-77% and liquid chromatography area percents (LCAP) >99% were achieved. The 2.5 L scale run lasted a total of 6 hours, producing 106.0 g; therefore, a throughput of 0.42 kg/day is achievable with this set-up. To in-crease throughput further, it would only be needed to increase the reactor sizes of the PFR, CSTR, and membrane separator. It was noticed that some crystallization occurred in-line after the membrane separation step and could lead to clogging after several hours of running. It is believed that gentle heating of the membrane separator and exit stream would prevent this. The new process eliminated the use of chloroform, bromoacetyl chloride, and high equivalency of NH3 while increasing the throughput and yield by 20-30% relative to our discovery route and reported continuous syntheses.

### Example 10. Imidophosphate Displacement

Initial studies for lactam activation and subsequent displacement with diethyl acetamidomalonate used diethyl chlorophosphate to give the desired product in good yield; however, there were concerns with using a known cholinesterase inhibitor at larger scales. Based on literature precedent, attention was directed to DMPC. The use of DMPC gave a slightly lower yield; however, DMPC is a crystalline solid and was safer to handle. DMPC was prepared from phosphorous oxychloride and morpholine in toluene—evolution of HCl necessitated 4 equivalents of morpholine and morpholine hydrochloride must be removed via filtration before the crude solution was concentrated and the product was crystallized with addition of anti-solvent. While DMPC worked reasonably well, sourcing DMPC was of concern as the process is transitioned from gram-scale to kilogram-scale processing. In addition, DMPC has a limited shelf-life and adventitious water resulted in the generation of morpholine hydrochloride. When older lots of DMPC were used, competition between morpholine and diethyl acetamidomalonate resulted in reduced yields. Thus, alternative reagents that were commercially available were identified and evaluated. Unsurprisingly, sub-stoichiometric equivalents of phosphorous oxychloride did not result in product formation; however, the use of diphenyl chlorophosphate resulted in complete consumption of the starting material to give the product in modest yield. Diphenyl chlorophosphate is a commercially available clear, viscous liquid with a variety of applications. Despite moisture sensitivity, it was hypothesized that the reduced nucleophilicity of phenol relative to morpholine would mitigate side-product formation and the phenol adduct was not observed. Reactions conducted with diphenyl chlorophosphate resulted in exceptionally thick mixtures as the reaction proceeded. On a small scale, the magnetic stir bar would seize, and no agitation was observed. Overhead stirring with an anchor impeller improved mixing in initial stages of the reaction but as the reaction progressed, only localized mixing near the shaft and impeller could be observed. An exhaustive survey of reaction parameters—alternative bases, alternative solvents, volumes of solvent, equivalents of base, equivalents of diethyl acetamidomalonate—resulted in little to no improvement in mixing, except for diglyme. It was hypothesized that phosphate salt by-products were the cause for the dense reaction mixture; addition of water to the crude reaction mixture dissolved the bulk of the mixture to give a light slurry and the product could be isolated via filtration. With the objective to scale the process to a 100-L reactor, alternative impeller geometries were evaluated. Both helical ribbon and ViscoJet designs improved mixing in the initial stages of the reaction but mixing near the walls of the vessel at end of reaction remained poor. Additional studies demonstrated good mixing was more important for early stages of the process i.e., deprotonation of the lactam and diethyl acetamidomalonate with potassium tert-butoxide and the process could tolerate non-ideal mixing after addition of the phosphorylating reagent.

### Example 11. Kraphco Decarboxylation

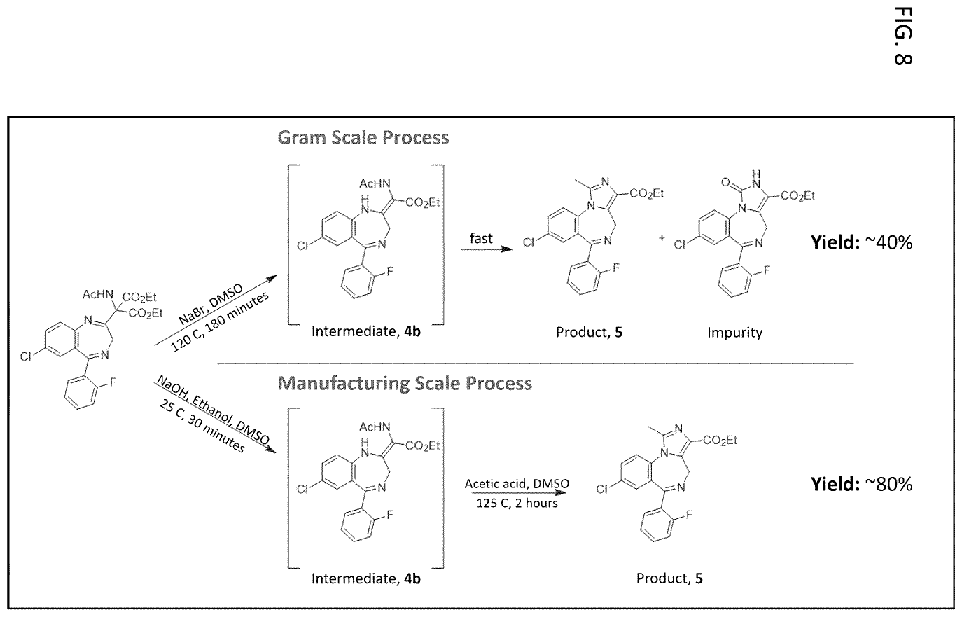

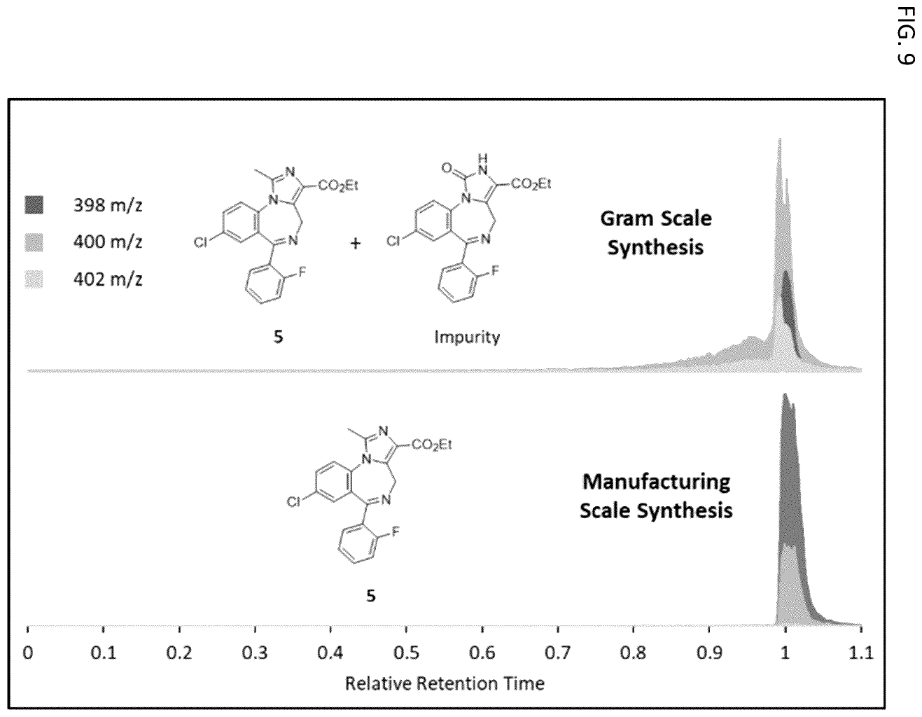

The krapcho decarboxylation and cyclization of the acetamidomalonate ethyl ester to form the imidazole was initially performed with NaBr in DMSO at elevated temperatures >130° C. The reaction passed through an olefin intermediate that would readily cyclize to the imidazole at elevated temperature but formed a significant impurity with a similar retention time but a m/z of 400, (+2 from the desired product). 1 H NMR analysis of isolated impurity showed a loss of the Imidazole methyl group, which leads to the hypothesis that the impurity is a cyclic urea, which could be formed by reaction of the olefin intermediate with the leaving CO₂ from the decarboxylation (Scheme of FIG. 8). Attempts to mitigate this biproduct via sparging were unsuccessful and resulted in the expulsion of haz-ardous ethyl bromide gas from solution. Moreover, the crude reaction mixture contained several baseline impurities, resulting in a poor crystallization that frequently resulted in oiling and low yields˜40%. We first explored other nucleophiles to eliminate the ethyl bromide gas, such as DBU and DABCO. These nucleophiles were able to perform the decarboxylation, however the impurity re-mained. We discovered that the decarboxylation reaction in EtOH with sub-stoichiometric amounts of NaOH resulted in clean decar-boxylation producing the olefin intermediate that did not readily cyclize. However, when solvent swapped to DMSO, the stable olefin intermediate would cyclize at 125° C. Therefore, decarboxylation was performed in a mixture of EtOH and DMSO, the hydroxide was quenched with acetic acid, then the reaction mixture was heated to distill off the ethanol, allowing the cyclization to proceed at 125° C. At this point the CO₂ in the reaction had been purged and the urea impurity was no longer detected by LCMS (FIG. 9). The lack of impurity could be further confirmed by the lack of the 402 m/z peak resulting from the isotope chlorine-37 that occurs in a 24% abundance. This adjustment in the process improved LCAP purity of the crude reaction from to 98%. It is believed that the use of protic solvent prevents the cyclization reaction. During the early stages of the process development, it was determined that the second step needed to be heated to 125° C. for the olefin intermediate to cyclize. NMR analysis was used to determine the solvent ratio of EtOH: DMSO during distillation, and these results were compared to LCMS analysis of the reaction. It was found that conversion did not start until a EtOH:DMSO ratio of 1:7 was achieved, and full conversion was not reached until the distillation temperature reached 125° C. The removal of ethanol by distillation was accelerated via nitrogen sparge, and a condenser was used to retain the DMSO solvent.

The improvement in the impurity profile allowed for a more aggressive crystallization that was cooled to −10 C in a DMSO:H₂O mixture. This process was scaled to a 20-L reactor for the manufacturing scale, allowing for a batch size of 4-kg of starting material. The improvements in synthesis and in purification culminated in nearly doubling the yield to 78-84%.

### Example 12. Ethyl Ester Saponification

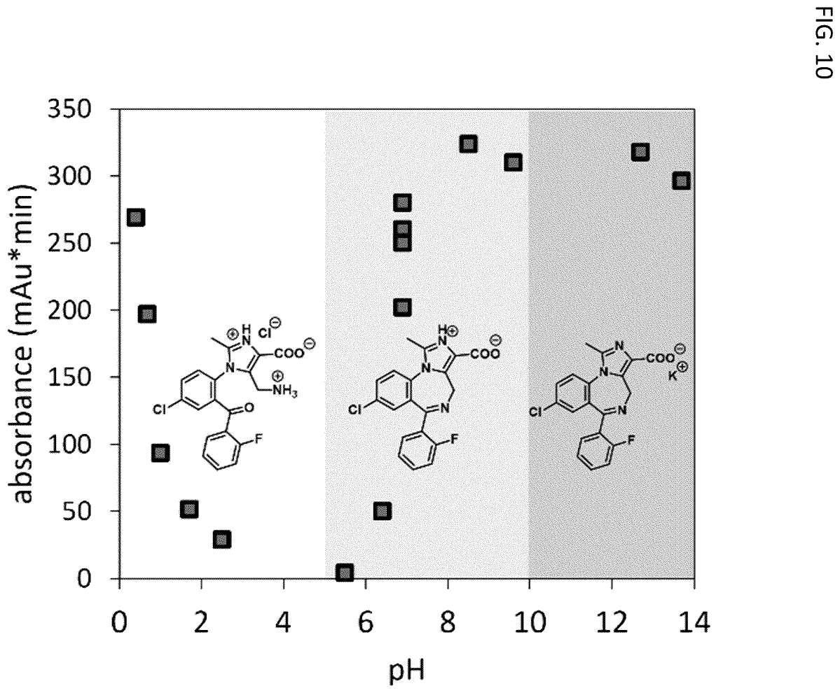

Saponification of midazolam ethyl ester to the carboxylic acid was performed both in batch and flow, but poor solubility limited the throughput of a continuous manufacturing approach. Therefore, the reaction was run as a batch in a methanol/KOH solution. Much of the R&D on this process step revolved around the reactive crystallization of the carboxylic acid product. Due to the amphoteric nature of the molecule, it was found that the product could be crystallized in various forms over a wide range of pH values. An initial screen was performed by titrating a batch of the carboxylate and analyzing the mother liquor at decreasing pH values to determine the pH value with the highest yield (FIG. 10).

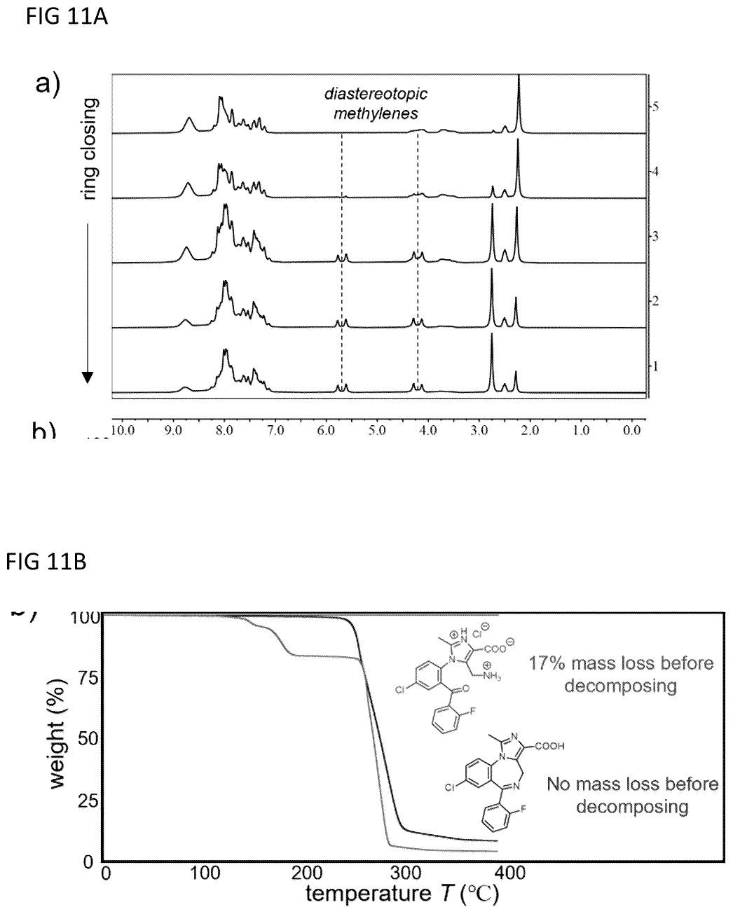

The titration experiment determined that pH 5.5-6.0 would provide the highest yielding crystallization. At pH values lower than this, the solubility of the product increases again until it is fully soluble at pH values below 1. This suggests that at these low pH values a different structure dominates in solution, the keto-ammonium, or “ring open” form. Upon standing overnight these low pH samples crystallized, and 1H NMR analysis revealed the open ring structure by identifying the absence of the diastereomeric methylene protons of the lactam ring. This was an attractive development as the ring open form had been previously reported to provide a purer decarboxylation. The ring quickly closes in neutral solvent, and the diastereomeric methylene resonances appear clearly between 4-6 ppm. This result suggests the ability to control the structure of Midazolam carboxylic acid as either a ring open or ring closed form by altering the crystallization pH. To test this hypothesis, we performed three batch reactions and crystallization at pH 1, and 5.5. The solid products were characterized by thermogravimetric analysis and ¹H NMR spectroscopy (FIGS. 11A and 11B).

Analysis of the TGA plot for the pH 1 crystallized solids showed two mass loss events between 100-200° C. (˜5% and ˜12% loss) followed by stability until decomposition at 280° C. The pH 5 solids did not have any mass loss until decomposition at the same temperature as the pH 1 solids, suggesting they are the same structure before decomposition. By using the % weight loss and the final molecular weight of closed form of 370 g/mol, a molecular weight of 443 g/mol was calculated for the pH 1 solids. It was thus postulate that these solids are the ring open HCl salt, with one water of hydration. Surprisingly, neither form readily decarboxylated during the experiments. Both solid forms were then used for the API step (Example 13), of decarboxylation to Midazolam. As will be discussed below, however, it was found that the ring open form did not result in a higher purity decarboxylation. Therefore, for the manufacturing route, it was decided to scale up the pH 5.5 crystallization, as the crystallization yield was higher. Batches were performed in a 20 L reactor and synthesized ˜1 kg per batch, with yields ranging from 95-99%.

### Example 13. Decarboxylation Reaction Development

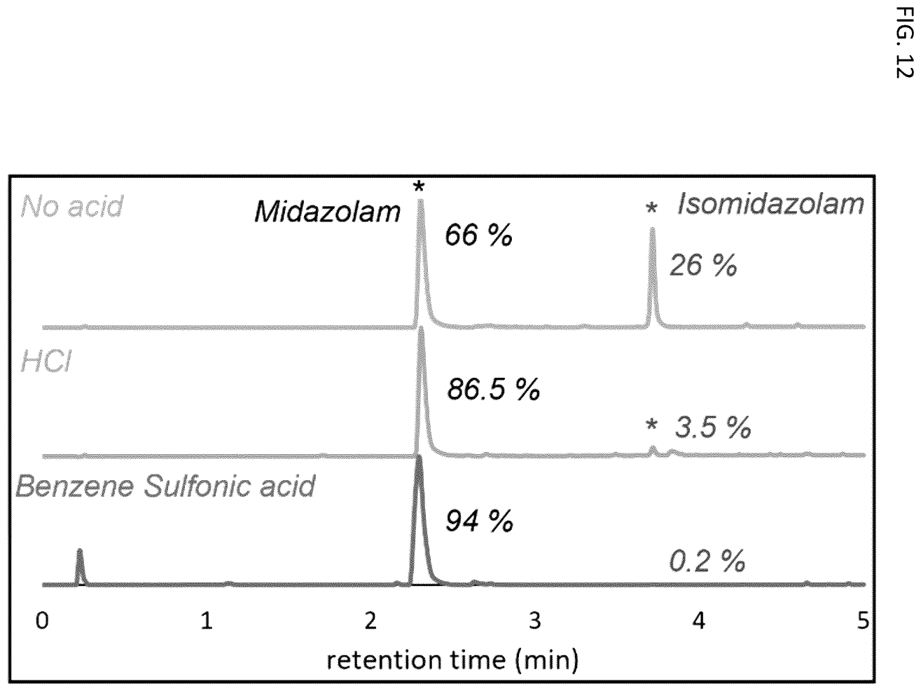

Historically, synthesis of midazolam via debcarboxylation has resulted in significant formation of isomidazolam, a structural isomer impurity that is difficult to purge, and must be controlled below 0.1% LCAP, per USP guidelines. The isomerization most likely occurs due to the high temperatures for decarboxylation of the imidazole, ranging from 180-230° C. Patent literature has suggested HCl can mitigate this formation, therefore our discovery route was an acidic decarboxylation in HCl/NMP at 180° C. This reaction provided midazolam with crude LCAP of 86.5% and isomidazolam of 3.5%, a midazolam:Isomidazolam ratio of ˜25:1. The same reaction without HCl produced isomidazolam at 26% LCAP, a 2.5:1 ratio. This result confirms that the presence of acid is important in mitigating the isomerization. Because the reaction temperature is much higher than the boiling point of HCl, it was thought that using a higher boiling acid could potentially aid in minimizing the isomidazolam impurity. Benzenesulfonic acid (BSA) is a solid acid with a low melting point of 50° C. and a boiling point of 190° C.; therefore it was envisioned that this solid acid could be used as a reagent and solvent in its liquid form. To do this, midazolam carboxylic acid was mixed with BSA in a 1:2.5 w:w ratio in a 20 mL vial and heated to reaction temperature. The solid acid melted and completely dissolved the starting material at 110° C. The melt was viscous but was able to be easily poured into other vials while maintaining temperature. At temperatures between 190-210° C. the decarboxylation reaction occurred, and LC analysis of the crude sample showed a midazolam LCAP of 94% (open ring+close ring), with an isomidazolam LCAP of only 0.2%, resulting in a ˜470:1 ratio of midazolam to isomidazo-lam (FIG. 12).

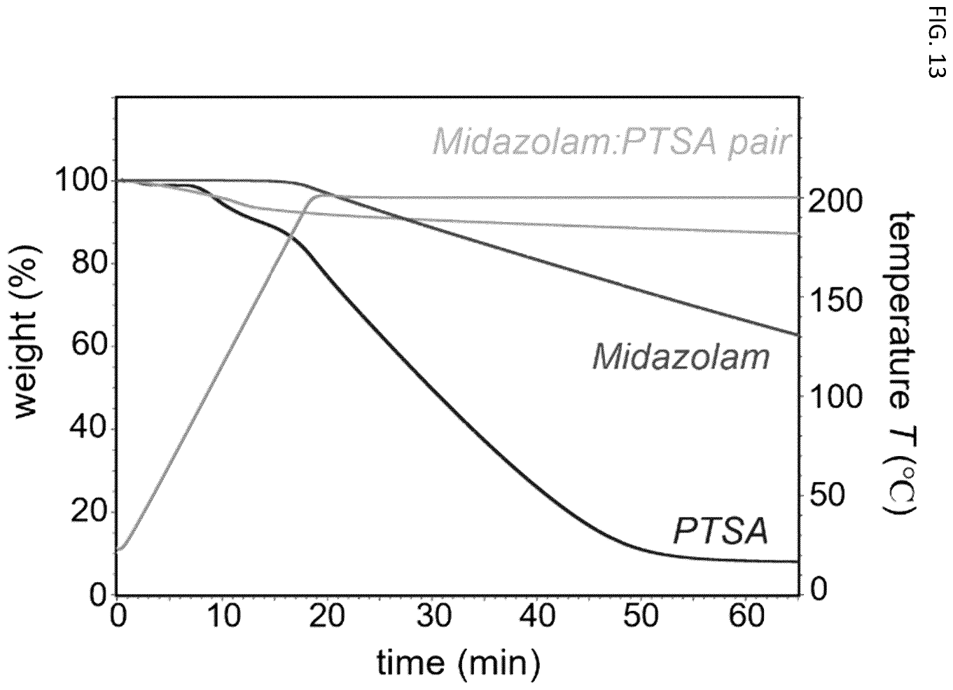

It appeared that the benzenesulfonic acid almost completely suppressed the formation of isomidazolam and resulted in higher midazolam LCAP. Without being bound by theory, an initial hypothesis is that a Bronsted acid protonates the imine/amine, forming a stable ion pair and prevents the isomerization of the imine at high temperatures. Due to the high boiling-point of BSA, the ion pair remains even at the high temperatures required for the decarboxylation. Moreover, it was determined that this strategy worked for other sulfonic acids with similar efficiency, such as para-toluenesulfonic acid (PTSA) and methanesulfonic acid (MSA). Due to the lower cost and similar performance of PTSA, it was decided to pursue this acid for the manufacturing route. Increasing mass ratios of PTSA:midazolam carboxylate were tested, and higher than 2.5:1 only increased purity minimally but required longer reactions time. Therefore, a 2.5:1 w:w ratio of PTSA:midazolam carboxylate was selected as the a reaction condition. To test the stable ion-pair hypothesis, TGA of PTSA, midazolam, and their combined salt pair were heated at 200° C. for 50 minutes and their decomposition rates were compared (FIG. 13). Upon heating, PTSA rapidly decomposed to ˜10 weight % within 40 minutes of heating. Midazolam decomposed linearly to 65% weight percent over the course of 50 min. LC-MS analysis of the TGA sample of midazolam showed the formation of isomidazolam with an LCAP of 12%. The midazolam:PTSA pair remained stable, and ended at 90 weight % of starting sample. LC-MS of this sample revealed only 1% LCAP isomidazolam was formed. The LCMS and TGA data suggest the ion pair interaction stabilizes midazolam and facilitates high temperature decarboxylation while inhibiting thermal isomerization to isomidazolam.

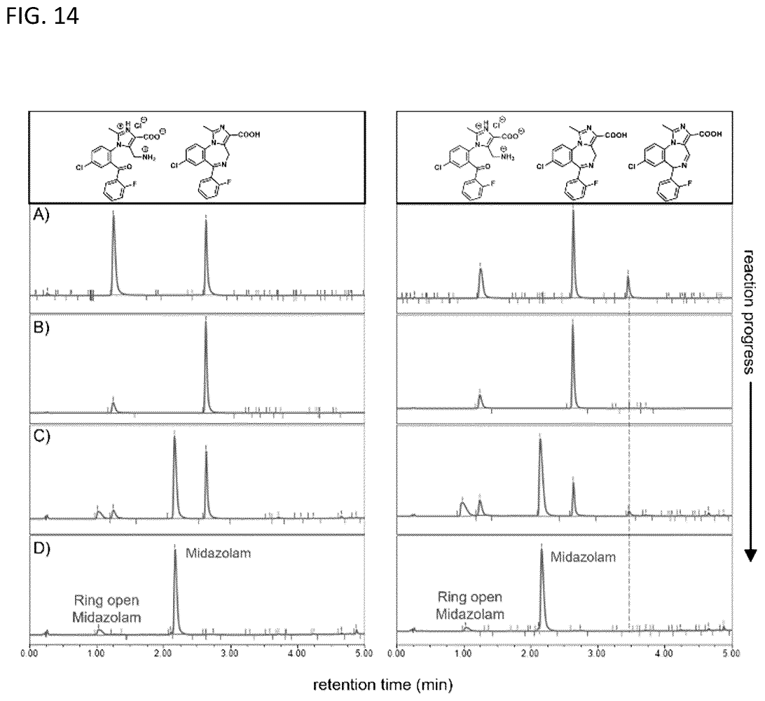

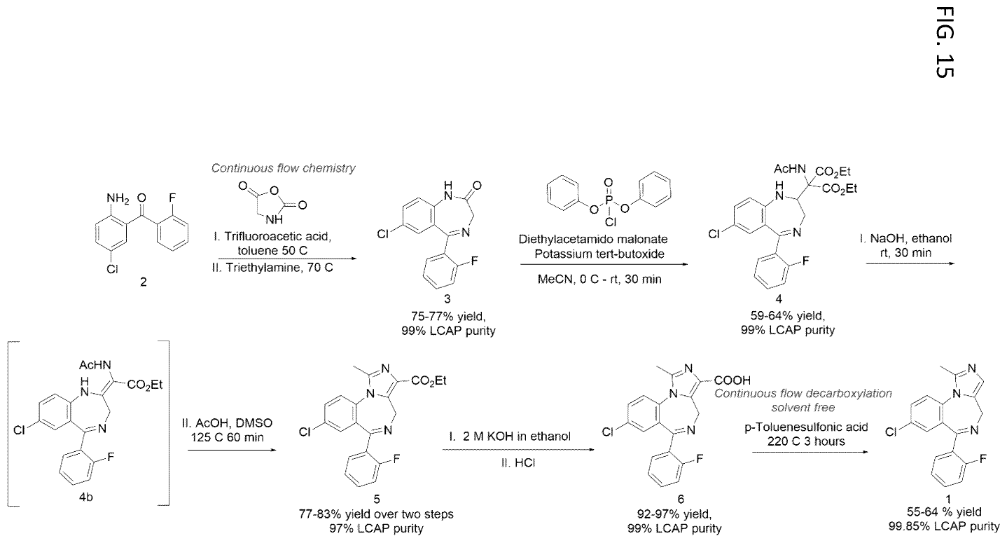

It was then decided to explore scale up and purification of this reaction to the manufacturing scale of 1 kg. First, it was needed to determine which of the two midazolam carboxylic acid structures should be used, the ring open or ring closed form. Comparatively, both ring open and ring closed forms produced crude midazolam reaction mixtures with similar impurity profiles, and at high temperatures the ring open form converts to ring closed before decarboxylation, agreeing with the TGA results in FIG. 13. Moreover, it was discovered that isomidazolam carboxylate present in the ring closed solids can be converted to the non-iso form by stirring in melted PTSA at lower than reaction temperatures, and then decarboxylated to midazolam by heating to >200° C. (FIG. 14).

This data led to choosing the ring closed solid form of midazolam carboxylate for the manufacturing route, as the isolation of this form was higher yielding. Due to the large amount of heat required for the melt reaction, it was deemed that continuous chemistry would be appropriate to increase the scale of this step.

In summary, a new manufacturing route to critical care medicine Midazolam that utilizes continuous chemistry and demonstrated on the kilogram scale was developed (FIG. 16). A novel melting decarboxylation reaction that nearly completely prevents the formation of Isomidazolam via formation of a stable ion-pair species was also demonstrated. The melt reaction was demonstrated on a continuous platform, by using an in-house built plate reactor that ran for an extended 24 hour run and produced nearly half a kilogram of midazolam in a hood-scale set-up.

What is claimed is:

1. A continuous flow process for synthesizing a compound of formula XVII, 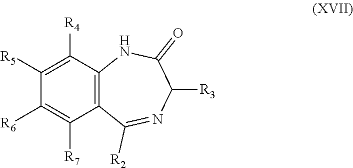
the process comprising the steps of: <a. reacting Glycine-N-Carboxyanhydride (Gly-NCA) with a phenone compound of formula XVIII in an acidic organic solution in a plug flow reactor; 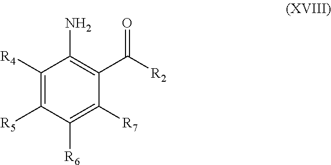
b. basifying the reaction product of a) with a Lewis base in a stirred tank reactor to thereby result in lactam cyclization and formation of the compound of formula XVII;
wherein:
R₂ and R₄-R₇ are each independently selected from the group consisting of H, a halogen, an alkyl, an aryl, and a heteroaryl, wherein the aryl and the heteroaryl are each independently unsubstituted or are optionally independently substituted with one or more groups selected from the group consisting of alkyl, alkenyl, alkynyl, alkoxyl, alkylsulfo, alkylamino, halogen, thiol, hydroxyl, nitro, cyano, cycloalkyl, heterocyclic alkyl, aryl, heteroaryl, cycloalkoxyl, heterocyclic alkoxyl, cycloalkylthio, and heterocyclic alkylthio.

2. The process of claim 1, wherein the acid organic solution comprises a non-polar solvent comprising an organofluorine acid.

3. The process of claim 1, wherein the acid organic solution comprises toluene comprising trifluoroacetic acid (TFA).

4. The process of claim 1, wherein Lewis base is triethylamine (TEA).

5. The process of claim 1 further comprising washing the reaction mixture with saturated aqueous alkali bicarbonate solution.

6. The process of claim 5, wherein the saturated aqueous alkali bicarbonate solution is saturated aqueous sodium bicarbonate.

7. The process of claim 5 further comprising separating organic and inorganic fractions of the reaction mixture.

8. The process of claim 7 wherein separating the organic and inorganic fractions of the reaction mixture comprises passing the reaction mixture through a membrane separator.

9. The process of claim 8, wherein the membrane separator is a Zaiput membrane separator with an OB 900 filter.

10. The process of claim 7, further comprising crystallizing the compound of formula XVII by pumping the organic fraction into a crystallizer comprising n-heptane (anti-solvent).

11. A process for synthesizing a compound of formula XIX, 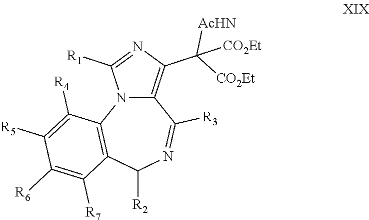
comprising the steps of:
a. mixing a compound of formula XVII 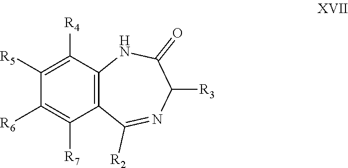
with a sufficient quantity of a polar, water miscible solvent;
b. adding diethylacetamidomalonate, potassium tert-butoxide, and diphenylphosphorylchloride (DPPC) or dimorpholinophosphinyl chloride (DMPC); and
c. allowing the reaction to proceed a sufficient period of time to produce the compound of formula XIX;
wherein:
R₁-R₇ are each independently selected from the group consisting of H, a halogen, an alkyl, an aryl, and a heteroaryl, wherein the aryl and the heteroaryl are each independently unsubstituted or are optionally independently substituted with one or more groups selected from the group consisting of alkyl, alkenyl, alkynyl, alkoxyl, alkylsulfo, alkylamino, halogen, thiol, hydroxyl, nitro, cyano, cycloalkyl, heterocyclic alkyl, aryl, heteroaryl, cycloalkoxyl, heterocyclic alkoxyl, cycloalkylthio, and heterocyclic alkylthio.

12. The process of claim 11, wherein the compound of formula XVII is mixed with the polar, water miscible solvent in a reactor at a temperature of about 0° C., with stirring.

13. The process of claim 11, wherein about two moles of potassium tert-butoxide are added.

14. The process of claim 11, wherein about 0.7 mole of DPPC is added.

15. The process of claim 11, wherein the reaction is incubated at 0° C. for about 1 hour.

16. The process of claim 11, further comprising crystallizing the compound of formula XIX by the steps of
a. inducing crystallization of the compound of formula XIX by the addition of an acid;
b. allowing the crystallized compound of formula XIX to precipitate; and
c. collecting the precipitate by filtration.

17. The process of claim 16, further comprising the steps of:
a. washing the filtered precipitate of with a mixture of isopropanol and water (1:1); and
b. drying the precipitate.

18. A process for making a compound of formula XX; 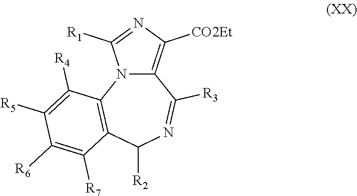
comprising the steps of:
a. mixing the compound of formula XIX with a sufficient quantity of the following: dimethyl sulfoxide (DMSO), an alcohol, and an alkali hydroxide and allowing the reaction to proceed for a period of time sufficient to synthesize the compound of formula XX;
b. quenching the alkali hydroxide with a sufficient amount of acid; and
c. heating the reaction mixture for a sufficient amount of time and a temperature to distill the alcohol off;
wherein:
R₁-R₇ are each independently selected from the group consisting of H, a halogen, an alkyl, an aryl, and a heteroaryl, wherein the aryl and the heteroaryl are each independently unsubstituted or are optionally independently substituted with one or more groups selected from the group consisting of alkyl, alkenyl, alkynyl, alkoxyl, alkylsulfo, alkylamino, halogen, thiol, hydroxyl, nitro, cyano, cycloalkyl, heterocyclic alkyl, aryl, heteroaryl, cycloalkoxyl, heterocyclic alkoxyl, cycloalkylthio, and heterocyclic alkylthio.

19. The process of claim 18, wherein the alkali hydroxide is sub-stoichiometric amounts of NaOH.

20. The process of claim 18, wherein the alcohol is ethanol.

21. The process of claim 18, wherein distillation is performed at 125° C. or more.

22. The process of claim 18 further comprising crystallizing the compound of formula XX by adding a sufficient quantity of water and cooling the reaction mixture with the addition of one or more seed crystals to create a crystallization slurry and collecting the crystallized compound of formula XX.

23. A process for making a compound of formula XXI comprising the steps of: 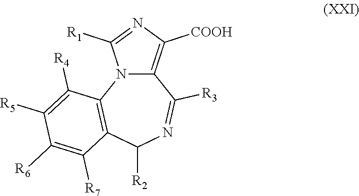
a. mixing a compound of formula XX with an alcohol; 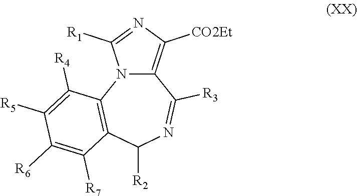
b. adding to the mixture of a) a solution of aqueous alkali hydroxide;
c. acidifying the solution of b) with the addition of a sufficient quantity of inorganic acid with stirring to precipitate the compound of formula XXI resulting in a slurry; and
d. collecting the crystals formed in c) by filtration and washing with a solution of about 1:1 acetone:water;
wherein:
R₁-R₇ are each independently selected from the group consisting of H, a halogen, an alkyl, an aryl, and a heteroaryl, wherein the aryl and the heteroaryl are each independently unsubstituted or are optionally independently substituted with one or more groups selected from the group consisting of alkyl, alkenyl, alkynyl, alkoxyl, alkylsulfo, alkylamino, halogen, thiol, hydroxyl, nitro, cyano, cycloalkyl, heterocyclic alkyl, aryl, heteroaryl, cycloalkoxyl, heterocyclic alkoxyl, cycloalkylthio, and heterocyclic alkylthio.

24. A process for the preparation of 4H-imidazo[1,5-a][1,4]benzodiazepine compounds of formula IX or a salt thereof; 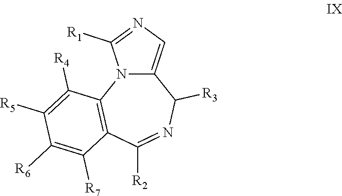
the process comprising the steps of:
a. mixing a compound of formula X, a compound of formula XI, a compound of formula XII, or any combination thereof with about 3 to about 7 molar equivalents of a sulfonic acid to form a reaction mixture, wherein the compound of formula XI is a 6H-imidazo[1,5-a][1,4]benzodiazepine isomer of the compound of formula IX and the compounds of formula XII is a 6H-imidazo[1,5-a][1,4]benzodiazepine isomer of the compound of formula X; 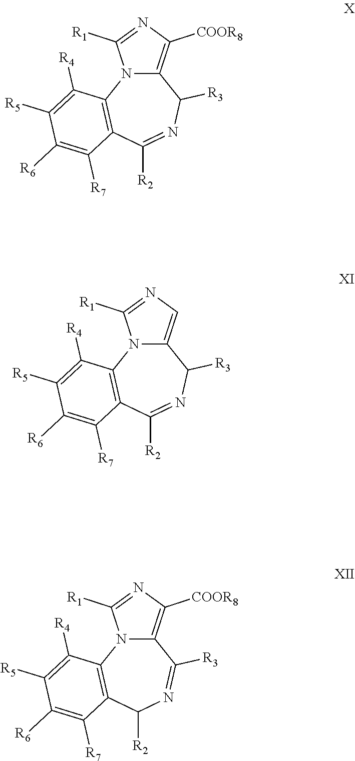
b. if the reaction mixture of a) comprises a compound of formula XI, a compound of formula XII, or any combination thereof, converting the 6H-imidazo[1,5-a][1,4]benzodiazepine isomer of formula XI to the 4H-imidazo[1,5-a][1,4]benzodiazepine isomer of formula IX to thereby synthesize the compound of formula IX and converting the 6H-imidazo[1,5-a][1,4]benzodiazepine isomer of formula XII to the 4H-imidazo[1,5-a][1,4]benzodiazepine isomer of formula X by heating the reaction mixture to a temperature ranging from about 130° C. to about 160° C. for a period of time ranging from about 60 minutes to about 120 minutes;
c. decarboxylating the 4H-imidazo[1,5-a][1,4]benzodiazepines of formula X and formula XII by heating any mixture from a) or b) comprising the 4H-imidazo[1,5-a][1,4]benzodiazepines of formula X and formula XII to a temperature ranging from about 170° C. to about 230° C. for a period of time ranging from about 30 minutes to about 240 minutes to thereby synthesize produce the 4H-imidazo[1,5-a][1,4]benzodiazepine of formula IX or a salt thereof;
wherein:
R₁-R₈ are each independently selected from the group consisting of H, a halogen, an alkyl, an aryl, and a heteroaryl, wherein the aryl and the heteroaryl are each independently unsubstituted or are optionally independently substituted with one or more groups selected from the group consisting of alkyl, alkenyl, alkynyl, alkoxyl, alkylsulfo, alkylamino, halogen, thiol, hydroxyl, nitro, cyano, cycloalkyl, heterocyclic alkyl, aryl, heteroaryl, cycloalkoxyl, heterocyclic alkoxyl, cycloalkylthio, and heterocyclic alkylthio.

25. The process of claim 24, wherein the 4H-imidazo[1,5-a][1,4]benzodiazepine compounds comprise compounds of formula XIII; 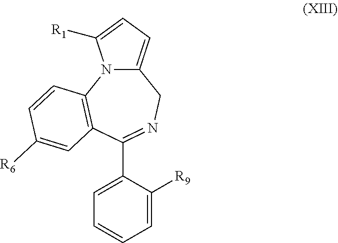
wherein the process comprises:
a. converting the 6H-imidazo[1,5-a][1,4]benzodiazepine isomer of formula XV to the 4H-imidazo[1,5-a][1,4]benzodiazepine isomer of formula XIII to thereby synthesize the compound of formula XIII and converting the 6H-imidazo[1,5-a][1,4]benzodiazepine isomer of formula XIV to the 4H-imidazo[1,5-a][1,4]benzodiazepine isomer of formula XVI; and
b. decarboxylating the 4H-imidazo[1,5-a][1,4]benzodiazepines of formula XIV and formula XVI to thereby synthesize produce the 4H-imidazo[1,5-a][1,4]benzodiazepine of formula XIII or a salt thereof 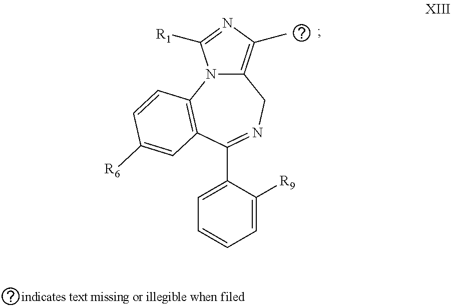
wherein:
R₁ is selected from the group consisting of H, —CH₃, —CH₂OH, and —C₂H₅; and
R₆ and R₉ are each independently selected from the group consisting of H and halogen; and
R₈ is selected from —COOH and —CO2Et.

26. The process of claim 24, wherein the 4H-imidazo[1,5-a][1,4]benzodiazepines comprise a compound of formula VI (midazolam); 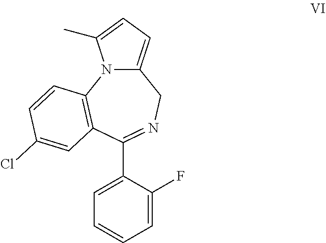 [SMILES: Cc1ccc2n1-c1ccc(Cl)cc1C(c1ccccc1F)=NC2]
wherein the 6H-imidazo[1,5-a][1,4]benzodiazepine isomer of midazolam (isomidazolam) is a compound of formula XXII 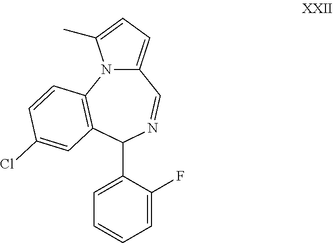 [SMILES: Cc1ccc2n1-c1ccc(Cl)cc1C(c1ccccc1F)N=C2]
and wherein the ratio of midazolam to isomidazolam is about 180:1 or greater.

27. The process of claim 24, wherein the sulfonic acid is benzenesulfonic acid, para-toluenesulfonic acid (PTSA), methanesulfonic acid (MSA), or any combination thereof.

28. The process of claim 24 further comprising purifying the 4H-imidazo[1,5-a][1,4]benzodiazepine compounds using the steps of:
a. adding a sufficient quantity of water to solubilize the 4H-imidazo[1,5-a][1,4]benzodiazepine of formula IX;
b. basifying the solution of a) with a sufficient quantity of concentrated aqueous alkali hydroxide solution;
c. extracting the solution of b) with a water-immiscible organic solvent at least two times, and collecting the organic layers;
d. treating the collected organic layers of c) with a sufficient quantity of activated charcoal for about 20 to 40 minutes;
e. filtering the charcoal treated organic solution through celite to remove the charcoal; and
f. concentrating the resultant solution of e) under vacuum to obtain the compound of formula IX as a solid.

29. The process of claim 24, further comprising purifying the 4H-imidazo[1,5-a][1,4]benzodiazepine compounds using the steps of:
a. adding a sufficient quantity of acetonitrile to solubilize the 4H-imidazo[1,5-a][1,4]benzodiazepine of formula IX;
b. cooling the solution of a) and seeding with crystals of the compound of formula IX;
c. collecting the resulting crystals by filtration; and
d. washing the crystals of c) in an isopropanol:n-heptane solution followed by n-heptane and drying the crystals.

30. A process for the for the preparation of 4H-imidazo[1,5-a][1,4]benzodiazepine compounds of formula IX or a salt thereof; 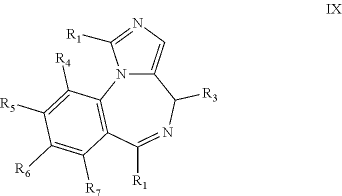
the process comprising combining one or more processes of claims 1-10, 11-17, 18-22, 23, or any combination thereof with the process of claims 24-29.

31. A process for the preparation of 4H-imidazo[1,5-a][1,4]benzodiazepine compounds of formula IX or a salt thereof; 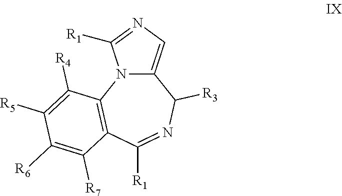
the process comprising combining one or more processes of claims 1-10, 11-17, 18-22, 23, and 24-29 in series.

32. A process for the for the preparation of 4H-imidazo[1,5-a][1,4]benzodiazepine compounds of formula IX or a salt thereof; 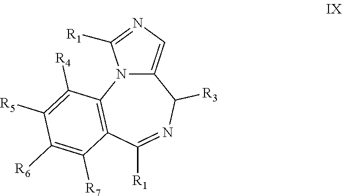
the process comprising combining one or more processes of claims 11-17, 18-22, 23, and 24-29 in series.

33. A process for the for the preparation of 4H-imidazo[1,5-a][1,4]benzodiazepine compounds of formula IX or a salt thereof; 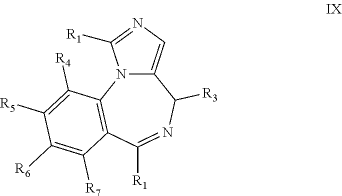
the process comprising combining one or more processes of claims 18-22, 23, and 24-29 in series.

34. A process for the for the preparation of 4H-imidazo[1,5-a][1,4]benzodiazepine compounds of formula IX or a salt thereof; 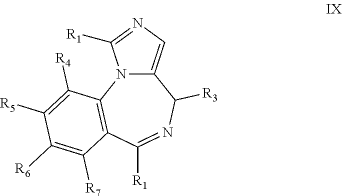
the process comprising combining one or more processes of claims 23, and 24-29 in series.

35. A process for making a compound of formula II: 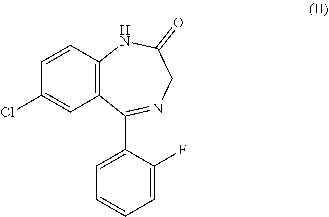 [SMILES: O=C1CN=C(c2ccccc2F)c2cc(Cl)ccc2N1]
the process having the steps of:
a. making a solution of toluene, trifluoracetic acid (TFA), and about 0.5 mole of 2-amino-5-chloro-2′-fluorobenzophenone;
b. stirring the mixture of a) and adding about 1.1 mole equivalents of glycine N-carboxyanhydride;
c. pumping the solution of b) into a plug flow reactor heated to about 50° C. with a flow rate of about 8 to 15 ml/min for about 10-20 minutes residence time;
d. converging the first outlet stream flow from c) with a second stream flow of triethylamine flowing at a rate of between about 0.7 to about 1.5 ml/min into a tank reactor heated to about 75° C. for about 90 minutes;
e. pumping the reaction solution of d) out of the reactor at a rate of about 10 to about 15 ml/min as a second outlet stream flow;
f. converging the second outlet stream flow of e) with a stream of saturated aqueous alkali bicarbonate solution at a flow rate of about 15 to about 25 ml/min;
g. separating the organic and inorganic fractions by combining the streams of f) into a membrane separator and collecting the organic layer created or in a gravity separator;
h. pumping the organic layer of g) into a crystallizer together with n-heptane (anti-solvent) at a flow rate of about 0.5 to 1.5 ml/min; and
i. collecting and drying the crystallized product.

36. A process for making a compound of formula Ill (midazolam-AFP) 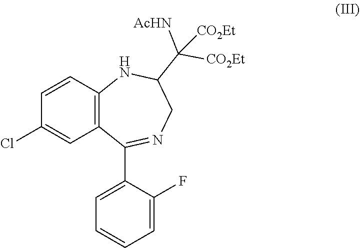 [SMILES: CCOC(=O)C(NC(C)=O)(C(=O)OCC)C1CN=C(c2ccccc2F)c2cc(Cl)ccc2N1]
comprising the steps of:
a. adding to a reactor at a temperature of about 0° C., a sufficient quantity of a polar, water miscible solvent with stirring;
b. adding to the solvent of a) about a mole of diethylacetamidomalonate, and about 0.6 mole of compound of formula II (midazolam-lactam or norflurazepam) to create a solution with stirring;
c. adding to the solution of b) about two moles of potassium tert-butoxide over a sufficient time to keep the temperature of the solution at about 0° C.;
d. adding to the solution of c) about 0.7 mole of diphenylphosphorylchloride (DPPC); and
e. allowing the reaction to proceed for about 1 hour.

37. The process of claim 36, wherein at step d) dimorpholinophosphinyl chloride (DMPC) is alternatively added.

38. The process of claim 36, further comprising the steps of determining the completeness of the reaction of e) by taking a sample of the solution of e) and measuring the quantity of yield of the compound of formula III in the solution of e).

39. The process of claim 38, further comprising the steps of
a. induce crystallization of the compound of formula III by adding a sufficient quantity of about a 5% acetic acid solution in water to the solution of e) to induce crystallization of the compound of formula III;
b. allowing the crystallized compound of formula III to precipitate or by adding a sufficient quantity of water to the solution of e); and
c. collecting the precipitate by filtration.

40. The process of claim 39, optionally comprising the steps of:
a. washing the filtered precipitate of i) with a mixture of isopropanol and water (1:1); and
b. drying the precipitate.

41. A process for making the compound of formula IV 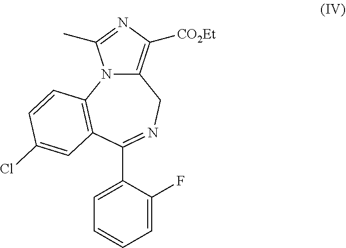 [SMILES: CCOC(=O)c1nc(C)n2c1CN=C(c1ccccc1F)c1cc(Cl)ccc1-2]
comprising the steps of:
a. adding about 2 moles of the compound of formula III into a reactor at a temp of about 25° C.;
b. adding to the reactor of a) a sufficient quantity of the following: dimethyl sulfoxide (DMSO), ethanol, and about 1.5 mol of alkali hydroxide, and stirring the resultant solution for about 30 to about 60 minutes;
c. optionally, determining the completeness of the reaction of b) by taking a sample of the solution of b) and measuring the quantity of yield of the intermediate formed midazolam-olefin intermediate (ethyl-2-acetamido-2-(7-chloro-5-(2-fluorophenyl)-1,3-dihydro-2H-benzo[e][1,4]diazepin-2-ylidene)acetate in the solution of b);
d. adding to the intermediate containing solution of b) a sufficient quantity, about 6 to 7 moles, of acetic acid and heating to 125° C.;
e. sparging the reactor with N₂ once the reactor reaches a temperature of at least about 70° C.;
f. stopping the N₂ sparging once the reactor reaches 125° C. and then stirring the reactor for between 30 to 60 minutes;
g. cooling the reactor to about 70° C.;
h. adding a sufficient quantity of deionized water and cooling the reactor to about 35° C. with the addition of one or more seed crystals to create a crystallization slurry;
i. cooling the slurry of h) to about -10° C. and stirring for at least about 30 min;
j. collecting the crystals of the compound of formula IV formed in step i) by filtration and washing with a sufficient quantity of deionized water; and
k. drying the crystals formed in j).

42. The process of claim 41, wherein at step b) an alkali ethoxide is substituted for an alkali hydroxide.

43. A process for making the compound of formula V comprising the steps of: 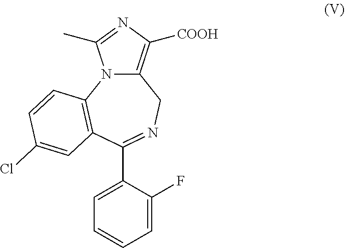 [SMILES: Cc1nc(C(=O)O)c2n1-c1ccc(Cl)cc1C(c1ccccc1F)=NC2]
a. adding to a reactor heated to about 50° C., a sufficient quantity of methanol with stirring;
b. adding to the methanol solution of a) about 1 to about 2 moles of the compound of formula IV and stirring the solution until the compound is dissolved;
c. adding to the solution of b) about 1 to 2 L of a about 2 to 3 molar aqueous alkali hydroxide solution and heating the reactor to about 70° C. for about 45 to 75 minutes;
d. optionally, determining the completeness of the reaction of c) by taking a sample of the solution of c) and measuring the quantity of yield of the compound of formula V formed in the solution of c);
e. acidifying the solution of c) with the addition of a sufficient quantity of 1M inorganic acid with stirring to precipitate the compound of formula V;
f. cooling the resulting slurry of e) to about room temp for about an hour and adjusting the pH of the slurry to about 5.2-5.7;
g. collecting the crystals formed in f) by filtration and washing with a solution of about 1:1 acetone:water; and
h. drying the crystals of the compound of formula V.

44. A process for making the compound of formula VI 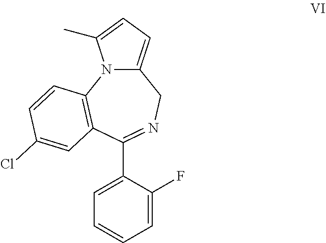 [SMILES: Cc1ccc2n1-c1ccc(Cl)cc1C(c1ccccc1F)=NC2]
comprising the steps of:
a. adding about 0.1 to 0.3 moles of the compound of formula V 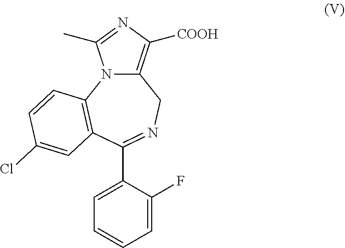 [SMILES: Cc1nc(C(=O)O)c2n1-c1ccc(Cl)cc1C(c1ccccc1F)=NC2]
to a container and adding about 5 to 6 mole equivalents of para-toluene sulfonic acid monohydrate and heating the container to about 150° C. for about 20 to 40 minutes to form a melted mixture;
b. heating the mixture of a) to 220° C. for about 2 to 3 hours to form a product; comprising the compound of formula VI;
c. cooling the reacted product of b) to about 100° C.;
d. adding to the mixture of c) a sufficient quantity of water to solubilize the product of c);
e. basifying the solution of d) with a sufficient quantity of concentrated aqueous alkali hydroxide solution;
f. extracting the solution of e) with a water-immiscible organic solvent at least two times, and collecting the organic layers;
g. treating the collected organic layers of f) with a sufficient quantity of activated charcoal for about 20 to 40 minutes;
h. filtering the charcoal treated organic solution through celite to remove the charcoal; and
i. concentrating the resultant solution under vacuum to obtain the compound of formula VI as a solid.

45. The process of claim 44, wherein at step a) the para-toluene sulfonic acid monohydrate is substituted by another sulfonic acid such as benzene sulfonic acid monohydrate, para-toluene sulfonic acid (anhydrous), benzene sulfonic acid (anhydrous) or methane sulfonic acid.

46. The process of 44, further comprising:
j) dissolving the solids of step i) in a sufficient quantity of acetonitrile and heating the resulting solution to about 70° C. to produce an optically clear solution;
k) cooling the solution of j) to about 60° C. and seeding with crystals of the compound of formula VI;
l) cooling the solution of k) to about −2° C. and held at this temperature for about 30 mins;
m) collecting the resulting crystals by filtration; and
n) washing the crystals of m) in about a 1:4 isopropanol:n-heptane solution followed by n-heptane and drying under vacuum.

47. The process of claim 44, wherein the process comprises:
j. at step d) diluting the cooled mixture of c) with water to a concentration of below about 40 mg of compound VI/ml;
k. filtering the solution of d) through a celite filter;
l. adding to the solution of e) about 6 mass equivalents (to midazolam) of acidic, neutral or basic alumina to create a slurry;
m. stirring the slurry of f) for about 1 to 2 hours and filtering out the alumina;
n. adding about 1/20 to ⅕ volumes of isopropanol and about 5 M aqueous alkali hydroxide is added to adjust the pH to greater than 10;
o. stirring the solution of h) for about 2 hours to allow the compound of formula VI to precipitate from the solution;
p. the resulting precipitate is redissolved in a sufficient quantity of 20% water in methanol and heated to about 40° C.;
q. the solution of j) is filtered to remove insoluble impurities and cooled to about 0° C. to allow crystallization;
r. collecting the resultant crystals from j) by filtration;
s. optionally washing the crystals with cold 20% water in methanol solution; and
t. drying the crystals of formula VI.

48. The process of claim 44, wherein the solvent at step f) is selected from mixtures of water/methanol, water/isopropanol, ethyl acetate/water, acetonitrile/water, or ethyl acetate/n-heptane.

49. A process for the preparation of midazolam comprising combining one or more processes of claims 35, 36-40, 41-42, 43, or any combination thereof with the process of claims 44-48.

50. A process for the preparation of midazolam comprising combining the process of each of claims 35, 36-40, 41-42, 43, and 44-48 in series.

51. A process for the preparation of midazolam comprising combining the process of each of claims 36-40, 41-42, 43, and 44-48 in series.

52. A process for the preparation of midazolam comprising combining the process of each of claims 41-42, 43, and 44-48 in series.

53. A process for the preparation of midazolam comprising combining the process of each of claims 43, and 44-48 in series.
